SEQUENCE LISTING (241 sequences)

SEQ ID NO: 1 - protein, 214 aa, Artificial Sequence
  anti-NCR3 Fab 72 Light chain
DIQMTQSPSSLSASVGDRVTITCRASQSVSSAVAWYQQKPGKAPKLLIYSASSLYSGVPSRFSGSRSGTDFTLTISSLQPEDFATYYCQQSSYWPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 10 aa, Artificial Sequence
  LCDR1
RASQSVSSAV

SEQ ID NO: 3 - protein, 7 aa, Artificial Sequence
  LCDR2
SASSLYS

SEQ ID NO: 4 - protein, 6 aa, Artificial Sequence
  LCDR3
SSYWPF

SEQ ID NO: 5 - protein, 236 aa, Artificial Sequence
  anti-NCR3 Fab 72 Heavy chain
EVQLVESGGGLVQPGGSLRLSCAASGFNISSSSIHWVRQAPGKGLEWVAYISSSSGYTSYADSVKGRFTISADTSKNTAYLQMNSLRAEDTAVYYCARYSYFYGGYFYWTSWGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 6 - protein, 6 aa, Artificial Sequence
  HCDR1
ISSSSI

SEQ ID NO: 7 - protein, 10 aa, Artificial Sequence
  HCDR2
YISSSSGYTS

SEQ ID NO: 8 - protein, 17 aa, Artificial Sequence
  HCDR3
YSYFYGGYFYWTSWGAF

SEQ ID NO: 9 - protein, 214 aa, Artificial Sequence
  anti-NCR3 Fab 78 Light chain
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 10 aa, Artificial Sequence
  LCDR1
RASQSVSSAV

SEQ ID NO: 11 - protein, 7 aa, Artificial Sequence
  LCDR2
SASSLYS

SEQ ID NO: 12 - protein, 6 aa, Artificial Sequence
  LCDR3
SSSSLI

SEQ ID NO: 13 - protein, 237 aa, Artificial Sequence
  anti-NCR3 Fab 78 Heavy chain
EVQLVESGGGLVQPGGSLRLSCAASGFNVSSSSIHWVRQAPGKGLEWVASISSSSGSTSYADSVKGRFTISADTSKNTAYLQMNSLRAEDTAVYYCARRISSYYMSYYDSFYYAGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 14 - protein, 6 aa, Artificial Sequence
  HCDR1
VSSSSI

SEQ ID NO: 15 - protein, 10 aa, Artificial Sequence
  HCDR2
SISSSSGSTS

SEQ ID NO: 16 - protein, 18 aa, Artificial Sequence
  HCDR3
RISSYYMSYYDSFYYAGM

SEQ ID NO: 17 - protein, 214 aa, Artificial Sequence
  anti-NCR3 Fab 79 Light chain
DIQMTQSPSSLSASVGDRVTITCRASQSVSSAVAWYQQKPGKAPKLLIYSASSLYSGVPSRFSGSRSGTDFTLTISSLQPEDFATYYCQQQWYPLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 10 aa, Artificial Sequence
  LCDR1
RASQSVSSAV

SEQ ID NO: 19 - protein, 7 aa, Artificial Sequence
  LCDR2
SASSLYS

SEQ ID NO: 20 - protein, 6 aa, Artificial Sequence
  LCDR3
QWYPLI

SEQ ID NO: 21 - protein, 231 aa, Artificial Sequence
  anti-NCR3 Fab 79 Heavy chain
EVQLVESGGGLVQPGGSLRLSCAASGFNVYSYSIHWVRQAPGKGLEWVASIYSYYGSTSYADSVKGRFTISADTSKNTAYLQMNSLRAEDTAVYYCARWYQYYYIGTAAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 22 - protein, 6 aa, Artificial Sequence
  HCDR1
VYSYSI

SEQ ID NO: 23 - protein, 10 aa, Artificial Sequence
  HCDR2
SIYSYYGSTS

SEQ ID NO: 24 - protein, 12 aa, Artificial Sequence
  HCDR3
WYQYYYIGTAAM

SEQ ID NO: 25 - protein, 214 aa, Artificial Sequence; identical to SEQ ID NO: 9
  anti-NCR1 Fab 77 Light chain

SEQ ID NO: 26 - protein, 10 aa, Artificial Sequence
  LCDR1
RASQSVSSAV

SEQ ID NO: 27 - protein, 7 aa, Artificial Sequence
  LCDR2
SASSLYS

SEQ ID NO: 28 - protein, 6 aa, Artificial Sequence
  LCDR3
SSSSLI

SEQ ID NO: 29 - protein, 237 aa, Artificial Sequence
  anti-NCR1 Fab 77 Heavy chain
EVQLVESGGGLVQPGGSLRLSCAASGFNVYYSYIHWVRQAPGKGLEWVASISSYYGSTYYADSVKGRFTISADTSKNTAYLQMNSLRAEDTAVYYCARSRYLQDYWSSWWVSWYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 30 - protein, 6 aa, Artificial Sequence
  HCDR1
VYYSYI

SEQ ID NO: 31 - protein, 10 aa, Artificial Sequence
  HCDR2
SISSYYGSTY

SEQ ID NO: 32 - protein, 18 aa, Artificial Sequence
  HCDR3
SRYLQDYWSSWWVSWYGL

SEQ ID NO: 33 - protein, 214 aa, Artificial Sequence
  anti-CD16 Fab 3 Light chain
DIQMTQSPSSLSASVGDRVTITCRASQSVSSAVAWYQQKPGKAPKLLIYSASSLYSGVPSRFSGSRSGTDFTLTISSLQPEDFATYYCQQSSAELITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 10 aa, Artificial Sequence
  LCDR1
RASQSVSSAV

SEQ ID NO: 35 - protein, 7 aa, Artificial Sequence
  LCDR2
SASSLYS

SEQ ID NO: 36 - protein, 6 aa, Artificial Sequence
  LCDR3
SSAELI

SEQ ID NO: 37 - protein, 238 aa, Artificial Sequence
  anti-CD16 Fab 3 Heavy chain
EVQLVESGGGLVQPGGSLRLSCAASGFNFSSYSIHWVRQAPGKGLEWVASIYSSSGSTSYADSVKGRFTISADTSKNTAYLQMNSLRAEDTAVYYCARWSYDQYYDQHGYYFYYWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 38 - protein, 6 aa, Artificial Sequence
  HCDR1
FSSYSI

SEQ ID NO: 39 - protein, 10 aa, Artificial Sequence
  HCDR2
SIYSSSGSTS

SEQ ID NO: 40 - protein, 19 aa, Artificial Sequence
  HCDR3
WSYDQYYDQHGYYFYYWGF

SEQ ID NO: 41 - protein, 18 aa, Artificial Sequence
  Summary of anti-NCR3 Fab 78 CDR H3 sequences
  MOD_RES (2)..(2): Xaa = any amino acid
  MOD_RES (3)..(3): Xaa = Gly, Ala, or Ser
  MOD_RES (5)..(5): Xaa = any amino acid
  MOD_RES (6)..(6): Xaa = Tyr or Phe
  MOD_RES (7)..(7): Xaa = any amino acid
  MOD_RES (8)..(8): Xaa = Ser or Thr
  MOD_RES (18)..(18): Xaa = Met or Leu
RXXSXXXXYYDSFYYAGX

SEQ ID NO: 42 - protein, 18 aa, Artificial Sequence
  HCDR3
RASSRFRSYYDSFYYAGM

SEQ ID NO: 43 - protein, 18 aa, Artificial Sequence
  HCDR3
RIGSIYRSYYDSFYYAGM

SEQ ID NO: 44 - protein, 18 aa, Artificial Sequence
  HCDR3
RISSHYMSYYDSFYYAGM

SEQ ID NO: 45 - protein, 18 aa, Artificial Sequence
  HCDR3
RISSSYMSYYDSFYYAGM

SEQ ID NO: 46 - protein, 18 aa, Artificial Sequence
  HCDR3
RISSYYISYYDSFYYAGM

SEQ ID NO: 47 - protein, 18 aa, Artificial Sequence
  HCDR3
RISSYYVSYYDSFYYAGM

SEQ ID NO: 48 - protein, 18 aa, Artificial Sequence
  HCDR3
RKSSSYWSYYDSFYYAGM

SEQ ID NO: 49 - protein, 18 aa, Artificial Sequence
  HCDR3
RKSSYYMSYYDSFYYAGM

SEQ ID NO: 50 - protein, 18 aa, Artificial Sequence
  HCDR3
RLGSRYRSYYDSFYYAGM

SEQ ID NO: 51 - protein, 18 aa, Artificial Sequence
  HCDR3
RRASYYKTYYDSFYYAGM

SEQ ID NO: 52 - protein, 18 aa, Artificial Sequence
  HCDR3
RRSSYYMTYYDSFYYAGM

SEQ ID NO: 53 - protein, 18 aa, Artificial Sequence
  HCDR3
RTGSYYMTYYDSFYYAGM

SEQ ID NO: 54 - protein, 18 aa, Artificial Sequence
  HCDR3
RTSSHYISYYDSFYYAGM

SEQ ID NO: 55 - protein, 18 aa, Artificial Sequence
  HCDR3
RVGSYYMSYYDSFYYAGM

SEQ ID NO: 56 - protein, 18 aa, Artificial Sequence
  HCDR3
RVSSNYMSYYDSFYYAGM

SEQ ID NO: 57 - protein, 18 aa, Artificial Sequence
  HCDR3
RVSSPYMSYYDSFYYAGL

SEQ ID NO: 58 - DNA, 642 bp, Artificial Sequence
  anti-NCR3 Fab 72 Light chain
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttcttactggccgttcacgttcggacagggtaccaaggtggagatcaaacgaactgtggctgcaccatctgtcttcatcttcccgccatctgattcacagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgt

SEQ ID NO: 59 - DNA, 708 bp, Artificial Sequence
  anti-NCR3 Fab 72 Heavy chain
gaggttcagctggtggagtctggcggtggcctggtgcagccagggggctcactccgtttgtcctgtgcagcttctggcttcaacatctcttcttcttctatacactgggtgcgtcaggccccgggtaagggcctggaatgggttgcatatatttcttcttcttctggctatacttcttatgccgatagcgtcaagggccgtttcactataagcgcagacacatccaaaaacacagcctacctacaaatgaacagcttaagagctgaggacactgccgtctattattgtgctcgctactcttacttctacggtggttacttctactggacttcttggggtgcttttgactactggggtcaaggaaccctggtcaccgtctcctcggcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacaca

SEQ ID NO: 60 - DNA, 642 bp, Artificial Sequence
  anti-NCR3 Fab 78 Light chain
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttcttcttctctgatcacgttcggacagggtaccaaggtggagatcaaacgaactgtggctgcaccatctgtcttcatcttcccgccatctgattcacagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgt

SEQ ID NO: 61 - DNA, 711 bp, Artificial Sequence
  anti-NCR3 Fab 78 Heavy chain
gaggttcagctggtggagtctggcggtggcctggtgcagccagggggctcactccgtttgtcctgtgcagcttctggcttcaacgtctcttcttcttctatacactgggtgcgtcaggccccgggtaagggcctggaatgggttgcatctatttcttcttcttctggctctacttcttatgccgatagcgtcaagggccgtttcactataagcgcagacacatccaaaaacacagcctacctacaaatgaacagcttaagagctgaggacactgcggtctattattgtgctcgccgtatctcttcttactacatgtcttactacgactctttctactacgctggtatggactactggggtcaaggaaccctggtcaccgtctcctcggcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacaca

SEQ ID NO: 62 - DNA, 642 bp, Artificial Sequence
  anti-NCR3 Fab 79 Light chain
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaacagtggtacccgctgatcacgttcggacagggtaccaaggtggagatcaaacgaactgtggctgcaccatctgtcttcatcttcccgccatctgattcacagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgt

SEQ ID NO: 63 - DNA, 693 bp, Artificial Sequence
  anti-NCR3 Fab 79 Heavy chain
gaggttcagctggtggagtctggcggtggcctggtgcagccagggggctcactccgtttgtcctgtgcagcttctggcttcaacgtctattcttattctatacactgggtgcgtcaggccccgggtaagggcctggaatgggttgcatctatttattcttattatggctctacttcttatgccgatagcgtcaagggccgtttcactataagcgcagacacatccaaaaacacagcctacctacaaatgaacagcttaagagctgaggacactgccgtctattattgtgctcgctggtaccagtactactacatcggtactgctgctatggactactggggtcaaggaaccctggtcaccgtctcctcggcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacaca

SEQ ID NO: 64 - DNA, 642 bp, Artificial Sequence; identical to SEQ ID NO: 60
  anti-NCR1 Fab 27 Light chain

SEQ ID NO: 65 - DNA, 711 bp, Artificial Sequence
  anti-NCR1 Fab 27 Heavy chain
gaggttcagctggtggagtctggcggtggcctggtgcagccagggggctcactccgtttgtcctgtgcagcttctggcttcaacgtctattattcttatatacactgggtgcgtcaggccccgggtaagggcctggaatgggttgcatctatttcttcttattatggctctacttattatgccgatagcgtcaagggccgtttcactataagcgcagacacatccaaaaacacagcctacctacaaatgaacagcttaagagctgaggacactgccgtctattattgtgctcgctctcgttacctgcaggactactggtcttcttggtgggtttcttggtacggtttggactactggggtcaaggaaccctggtcaccgtctcctcggcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacaca

SEQ ID NO: 66 - DNA, 642 bp, Artificial Sequence
  anti-CD16 Fab 3 Light chain
gatatccagatgacccagtccccgagctccctgtccgcctctgtgggcgatagggtcaccatcacctgccgtgccagtcagtccgtgtccagcgctgtagcctggtatcaacagaaaccaggaaaagctccgaagcttctgatttactcggcatccagcctctactctggagtcccttctcgcttctctggtagccgttccgggacggatttcactctgaccatcagcagtctgcagccggaagacttcgcaacttattactgtcagcaatcttctgctgaactgatcacgttcggacagggtaccaaggtggagatcaaacgaactgtggctgcaccatctgtcttcatcttcccgccatctgattcacagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgt

SEQ ID NO: 67 - DNA, 714 bp, Artificial Sequence
  anti-CD16 Fab 3 Heavy chain
gaggttcagctggtggagtctggcggtggcctggtgcagccagggggctcactccgtttgtcctgtgcagcttctggcttcaacttctcttcttattctatacactgggtgcgtcaggccccgggtaagggcctggaatgggttgcatctatttattcttcttctggctctacttcttatgccgatagcgtcaagggccgtttcactataagcgcagacacatccaaaaacacagcctacctacaaatgaacagcttaagagctgaggacactgccgtctattattgtgctcgctggtcttacgaccagtactacgaccagcatggttactacttctactactggggttttgactactggggtcaaggaaccctggtcaccgtctcctcggcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacaca

SEQ ID NO: 68 - DNA, 31 bp, Artificial Sequence
  primer
tgaggacactgccgtctattattgtgctcgc

SEQ ID NO: 69 - DNA, 59 bp, Artificial Sequence
  custom sequence
tactggggtcaaggaaccctggtcaagatcggaagagcacacgtctgaactccagtcac

SEQ ID NO: 70 - protein, 7 aa, Artificial Sequence
  CD16.01 LC3
YRGGRLI

SEQ ID NO: 71 - protein, 6 aa, Artificial Sequence
  CD16.01 HC1
VYSYYI

SEQ ID NO: 72 - protein, 10 aa, Artificial Sequence
  CD16.01 HC2
YISPSSGSTY

SEQ ID NO: 73 - protein, 12 aa, Artificial Sequence
  CD16.01 HC3
GYYSAWRYSMGL

SEQ ID NO: 74 - protein, 7 aa, Artificial Sequence
  CD16.02 LC3
AMYWELL

SEQ ID NO: 75 - protein, 6 aa, Artificial Sequence
  CD16.02 HC1
VSSSYI

SEQ ID NO: 76 - protein, 10 aa, Artificial Sequence
  CD16.02 HC2
SIYSYYGYTS

SEQ ID NO: 77 - protein, 18 aa, Artificial Sequence
  CD16.02 HC3
SYSYYSTWYMHRYFYAAF

SEQ ID NO: 78 - protein, 6 aa, Artificial Sequence
  CD16.05 LC3
WPGSLV

SEQ ID NO: 79 - protein, 6 aa, Artificial Sequence
  CD16.05 HC1
FYYYSI

SEQ ID NO: 80 - protein, 10 aa, Artificial Sequence
  CD16.05 HC2
SIYPSSGSTS

SEQ ID NO: 81 - protein, 6 aa, Artificial Sequence
  CD16.05 HC3
WSYHGM

SEQ ID NO: 82 - protein, 18 aa, Artificial Sequence
  CD16.06 HC3
FPYRWSYYWYSYYKWYAM

SEQ ID NO: 83 - protein, 7 aa, Artificial Sequence
  CD16.07 LC3
YGQRNPI

SEQ ID NO: 84 - protein, 6 aa, Artificial Sequence
  CD16.07 HC1
FYSSYI

SEQ ID NO: 85 - protein, 10 aa, Artificial Sequence
  CD16.07 HC2
SISPYSGYTY

SEQ ID NO: 86 - protein, 8 aa, Artificial Sequence
  CD16.07 HC3
SWYYFSAM

SEQ ID NO: 87 - protein, 6 aa, Artificial Sequence
  CD16.08 LC3
SSHHLI

SEQ ID NO: 88 - protein, 10 aa, Artificial Sequence
  CD16.08 HC2
SIYPSYGYTS

SEQ ID NO: 89 - protein, 7 aa, Artificial Sequence
  CD16.08 HC3
TYSYYAM

SEQ ID NO: 90 - protein, 6 aa, Artificial Sequence
  CD244.01 LC3
SVGVPV

SEQ ID NO: 91 - protein, 6 aa, Artificial Sequence
  CD244.01 HC1
LSYSSI

SEQ ID NO: 92 - protein, 12 aa, Artificial Sequence
  CD244.01 HC3
YYEYYGYGYAAM

SEQ ID NO: 93 - protein, 7 aa, Artificial Sequence
  CD244.02 LC3
SYNYSPL

SEQ ID NO: 94 - protein, 6 aa, Artificial Sequence
  CD244.02 HC1
ISYYSI

SEQ ID NO: 95 - protein, 10 aa, Artificial Sequence
  CD244.02 HC2
SIYPYYGYTS

SEQ ID NO: 96 - protein, 8 aa, Artificial Sequence
  CD244.02 HC3
NYYLYYGM

SEQ ID NO: 97 - protein, 7 aa, Artificial Sequence
  NCR1.01 LC3
SYYYDLV

SEQ ID NO: 98 - protein, 19 aa, Artificial Sequence
  NCR1.01 HC3
YKFKNAYWWSYYNTQGVGM

SEQ ID NO: 99 - protein, 7 aa, Artificial Sequence
  NCR1.02 LC3
SMYHQLI

SEQ ID NO: 100 - protein, 6 aa, Artificial Sequence
  NCR1.02 HC1
VSYSSI

SEQ ID NO: 101 - protein, 18 aa, Artificial Sequence
  NCR1.02 HC3
YSFEPSLYSYYWDSRSAF

SEQ ID NO: 102 - protein, 6 aa, Artificial Sequence
  NCR1.03 LC3
WWSSPI

SEQ ID NO: 103 - protein, 6 aa, Artificial Sequence
  NCR1.03 HC1
VSSYSI

SEQ ID NO: 104 - protein, 10 aa, Artificial Sequence
  NCR1.03 HC2
SIYSSYGSTY

SEQ ID NO: 105 - protein, 12 aa, Artificial Sequence
  NCR1.03 HC3
KMGWYYYWYDGL

SEQ ID NO: 106 - protein, 7 aa, Artificial Sequence
  NCR1.04 LC3
SYYYYLV

SEQ ID NO: 107 - protein, 6 aa, Artificial Sequence
  NCR1.04 HC1
FSYSYI

SEQ ID NO: 108 - protein, 10 aa, Artificial Sequence
  NCR1.04 HC2
SISSSSGYTS

SEQ ID NO: 109 - protein, 13 aa, Artificial Sequence
  NCR1.04 HC3
HYYRDFYQWTGGM

SEQ ID NO: 110 - protein, 6 aa, Artificial Sequence
  NCR1.05 LC3
SWAWPV

SEQ ID NO: 111 - protein, 6 aa, Artificial Sequence
  NCR1.05 HC1
FSYSSI

SEQ ID NO: 112 - protein, 6 aa, Artificial Sequence
  NCR1.05 HC3
FLGGAM

SEQ ID NO: 113 - protein, 6 aa, Artificial Sequence
  NCR1.06 LC3
SYFPPI

SEQ ID NO: 114 - protein, 6 aa, Artificial Sequence
  NCR1.06 HC1
LYSSSI

SEQ ID NO: 115 - protein, 10 aa, Artificial Sequence
  NCR1.06 HC2
SIYPYYSSTS

SEQ ID NO: 116 - protein, 14 aa, Artificial Sequence
  NCR1.06 HC3
SDGYWYWYGWYWGM

SEQ ID NO: 117 - protein, 6 aa, Artificial Sequence
  NCR1.07 LC3
STYEPI

SEQ ID NO: 118 - protein, 6 aa, Artificial Sequence
  NCR1.07 HC1
VYYSSI

SEQ ID NO: 119 - protein, 10 aa, Artificial Sequence
  NCR1.07 HC2
SISSYSSYTS

SEQ ID NO: 120 - protein, 18 aa, Artificial Sequence
  NCR1.07 HC3
EVHWYRYSYWYYYLTQAM

SEQ ID NO: 121 - protein, 7 aa, Artificial Sequence
  NCR1.08 LC3
NWYYPLI

SEQ ID NO: 122 - protein, 10 aa, Artificial Sequence
  NCR1.08 HC2
SISPYSGYTS

SEQ ID NO: 123 - protein, 16 aa, Artificial Sequence
  NCR1.08 HC3
MYNYQMYWLFGGGYGM

SEQ ID NO: 124 - protein, 7 aa, Artificial Sequence
  NCR1.09 LC3
SSGWYPL

SEQ ID NO: 125 - protein, 12 aa, Artificial Sequence
  NCR1.09 HC3
SESYWPYGWYGF

SEQ ID NO: 126 - protein, 6 aa, Artificial Sequence
  NCR1.10 HC1
LSSYSI

SEQ ID NO: 127 - protein, 10 aa, Artificial Sequence
  NCR1.10 HC2
SISSYYSSTS

SEQ ID NO: 128 - protein, 18 aa, Artificial Sequence
  NCR1.10 HC3
SWPYSSQQWYYYYYWYAM

SEQ ID NO: 129 - protein, 6 aa, Artificial Sequence
  CD16.06 HC1
FSSSSI

SEQ ID NO: 130 - protein, 17 aa, Artificial Sequence
  NCR1.12 HC3
SSMWSFPGGWQHYSFGM

SEQ ID NO: 131 - protein, 6 aa, Artificial Sequence
  NCR1.13 LC3
SSYSLV

SEQ ID NO: 132 - protein, 6 aa, Artificial Sequence
  NCR1.13 HC1
FYSSSI

SEQ ID NO: 133 - protein, 13 aa, Artificial Sequence
  NCR1.13 HC3
PWQPAYYGYAYGM

SEQ ID NO: 134 - protein, 6 aa, Artificial Sequence
  NCR1.14 HC1
VYSSSI

SEQ ID NO: 135 - protein, 10 aa, Artificial Sequence
  NCR1.14 HC2
SISPYYGYTS

SEQ ID NO: 136 - protein, 12 aa, Artificial Sequence
  NCR1.14 HC3
TGQYYYSYVYGL

SEQ ID NO: 137 - protein, 6 aa, Artificial Sequence
  NCR1.15 LC3
GYENPL

SEQ ID NO: 138 - protein, 6 aa, Artificial Sequence
  NCR1.15 HC1
VYSSYI

SEQ ID NO: 139 - protein, 10 aa, Artificial Sequence
  NCR1.15 HC2
SIYSSYGYTS

SEQ ID NO: 140 - protein, 16 aa, Artificial Sequence
  NCR1.15 HC3
YAFSLPSMWWYSYSGM

SEQ ID NO: 141 - protein, 6 aa, Artificial Sequence
  NCR1.16 LC3
SWYYPI

SEQ ID NO: 142 - protein, 16 aa, Artificial Sequence
  NCR1.16 HC3
SYVGRYSYYYYPGMGI

SEQ ID NO: 143 - protein, 6 aa, Artificial Sequence
  NCR1.17 LC3
SAHYPV

SEQ ID NO: 144 - protein, 10 aa, Artificial Sequence
  NCR1.17 HC2
SIYSSSSSTY

SEQ ID NO: 145 - protein, 17 aa, Artificial Sequence
  NCR1.17 HC3
EGLGWTWGAWLYWYGGI

SEQ ID NO: 146 - protein, 7 aa, Artificial Sequence
  NCR1.18 LC3
SGWSSPI

SEQ ID NO: 147 - protein, 10 aa, Artificial Sequence
  NCR1.18 HC2
SISPSYGYTS

SEQ ID NO: 148 - protein, 12 aa, Artificial Sequence
  NCR1.18 HC3
DILFFSYWYSAL

SEQ ID NO: 149 - protein, 6 aa, Artificial Sequence
  NCR1.19 LC3
SSYHPL

SEQ ID NO: 150 - protein, 17 aa, Artificial Sequence
  NCR1.19 HC3
WNTMYYYWYRPWGSWAM

SEQ ID NO: 151 - protein, 8 aa, Artificial Sequence
  NCR1.20 LC3
SWHSSSLI

SEQ ID NO: 152 - protein, 12 aa, Artificial Sequence
  NCR1.20 HC3
EYQQSYWYKWAL

SEQ ID NO: 153 - protein, 7 aa, Artificial Sequence
  NCR1.21 LC3
GWYFTLI

SEQ ID NO: 154 - protein, 10 aa, Artificial Sequence
  NCR1.21 HC2
SISSSYGSTS

SEQ ID NO: 155 - protein, 16 aa, Artificial Sequence
  NCR1.21 HC3
YPRSYFWGMVSRTQGL

SEQ ID NO: 156 - protein, 7 aa, Artificial Sequence
  NCR1.22 LC3
VRFGYPV

SEQ ID NO: 157 - protein, 18 aa, Artificial Sequence
  NCR1.22 HC3
SDLQQYWWWPSYYGYGGF

SEQ ID NO: 158 - protein, 6 aa, Artificial Sequence
  NCR1.23 LC3
SYVSPI

SEQ ID NO: 159 - protein, 10 aa, Artificial Sequence
  NCR1.23 HC2
YISSSYGYTY

SEQ ID NO: 160 - protein, 18 aa, Artificial Sequence
  NCR1.23 HC35
DMYKPGWYYYWGSGDYGF

SEQ ID NO: 161 - protein, 6 aa, Artificial Sequence
  NCR1.24 LC3
SYAWPV

SEQ ID NO: 162 - protein, 10 aa, Artificial Sequence
  NCR1.24 HC2
SISSYSGYTY

SEQ ID NO: 163 - protein, 16 aa, Artificial Sequence
  NCR1.24 HC3
NSWLRGTMSAYRYQGL

SEQ ID NO: 164 - protein, 6 aa, Artificial Sequence
  NCR1.25 LC3
YYFKPI

SEQ ID NO: 165 - protein, 10 aa, Artificial Sequence
  NCR1.25 HC2
SISSYSSSTY

SEQ ID NO: 166 - protein, 17 aa, Artificial Sequence
  NCR1.25 HC3
NHYYAQSYGIYQYFSGF

SEQ ID NO: 167 - protein, 7 aa, Artificial Sequence
  NCR3.01 LC3
SSYEYPV

SEQ ID NO: 168 - protein, 10 aa, Artificial Sequence
  NCR3.01 HC2
SIYPSSGSTY

SEQ ID NO: 169 - protein, 14 aa, Artificial Sequence
  NCR3.01 HC3
WYIKWDTWYYSDAM

SEQ ID NO: 170 - protein, 6 aa, Artificial Sequence
  NCR3.02 LC3
SYGSLI

SEQ ID NO: 171 - protein, 10 aa, Artificial Sequence
  NCR3.02 HC2
SIYPYYGSTS

SEQ ID NO: 172 - protein, 12 aa, Artificial Sequence
  NCR3.02 HC3
WYQYYYQYSLGF

SEQ ID NO: 173 - protein, 6 aa, Artificial Sequence
  NCR3.03 LC3
SYGFLI

SEQ ID NO: 174 - protein, 10 aa, Artificial Sequence
  NCR3.03 HC2
SIYPYSGYTS

SEQ ID NO: 175 - protein, 12 aa, Artificial Sequence
  NCR3.03 HC3
WYNYHHWWSRAF

SEQ ID NO: 176 - protein, 6 aa, Artificial Sequence
  NCR3.04 LC3
SGRQLV

SEQ ID NO: 177 - protein, 10 aa, Artificial Sequence
  NCR3.04 HC2
SIYSYYGSTY

SEQ ID NO: 178 - protein, 13 aa, Artificial Sequence
  NCR3.04 HC3
WYQYYPSQGGMAM

SEQ ID NO: 179 - protein, 7 aa, Artificial Sequence
  NCR3.05 LC3
SSWYYPF

SEQ ID NO: 180 - protein, 6 aa, Artificial Sequence
  NCR3.05 HC1
LYSYSI

SEQ ID NO: 181 - protein, 14 aa, Artificial Sequence
  NCR3.05 HC3
WYQYWDWSYSRGAM

SEQ ID NO: 182 - protein, 6 aa, Artificial Sequence
  NCR3.06 LC3
FSLSLL

SEQ ID NO: 183 - protein, 8 aa, Artificial Sequence
  NCR3.06 HC3
SYSYGFAM

SEQ ID NO: 184 - protein, 8 aa, Artificial Sequence
  NCR3.07 LC3
SFGFYYPF

SEQ ID NO: 185 - protein, 18 aa, Artificial Sequence
  NCR3.07 HC3
DYTSNSYGDYYYYGGYGF

SEQ ID NO: 186 - protein, 7 aa, Artificial Sequence
  NCR3.08 LC3
SGYYWPI

SEQ ID NO: 187 - protein, 18 aa, Artificial Sequence
  NCR3.08 HC3
WLMWFSYAHGAYHMPYGL

SEQ ID NO: 188 - protein, 6 aa, Artificial Sequence
  NCR3.09 LC3
SSKYLV

SEQ ID NO: 189 - protein, 10 aa, Artificial Sequence
  NCR3.09 HC2
YIYPYSGSTS

SEQ ID NO: 190 - protein, 12 aa, Artificial Sequence
  NCR3.09 HC3
WFTYHWPGSIAF

SEQ ID NO: 191 - protein, 10 aa, Artificial Sequence
  NCR3.10 HC3
WYQYSDSIAM

SEQ ID NO: 192 - protein, 6 aa, Artificial Sequence
  NCR3.13 LC3
YYGYLI

SEQ ID NO: 193 - protein, 6 aa, Artificial Sequence
  NCR3.14 HC1
FYSYSI

SEQ ID NO: 194 - protein, 18 aa, Artificial Sequence
  NCR3.14 HC3
YFYDSPYYTYFSNYPSAL

SEQ ID NO: 195 - protein, 19 aa, Artificial Sequence
  NCR3.15 HC3
YVGPSYHSVWYYYSWYYAI

SEQ ID NO: 196 - protein, 6 aa, Artificial Sequence
  NCR3.16 LC3
SWESLV

SEQ ID NO: 197 - protein, 18 aa, Artificial Sequence
  NCR3.16 HC3
YHDRYTYYFGYYGWSYGM

SEQ ID NO: 198 - protein, 7 aa, Artificial Sequence
  NCR3.17 LC3
HWRVSLI

SEQ ID NO: 199 - protein, 10 aa, Artificial Sequence
  NCR3.17 HC2
SIYSYSGSTS

SEQ ID NO: 200 - protein, 13 aa, Artificial Sequence
  NCR3.17 HC3
WYMYYYNSDYFGL

SEQ ID NO: 201 - protein, 19 aa, Artificial Sequence
  NCR3.20 HC3
RYKGIVWWSYWSNWYYMGL

SEQ ID NO: 202 - protein, 6 aa, Artificial Sequence
  NCR3.21 LC3
KGNRLI

SEQ ID NO: 203 - protein, 6 aa, Artificial Sequence
  NCR3.21 HC1
ISYSSI

SEQ ID NO: 204 - protein, 10 aa, Artificial Sequence
  NCR3.21 HC2
SIYSYYSSTS

SEQ ID NO: 205 - protein, 18 aa, Artificial Sequence
  NCR3.21 HC3
YSGGYFLFYTYDYYVYGF

SEQ ID NO: 206 - protein, 12 aa, Artificial Sequence
  NCR3.22 HC3
WYGYYYYGEFAF

SEQ ID NO: 207 - protein, 6 aa, Artificial Sequence
  NCR3.23 LC3
SSYGLF

SEQ ID NO: 208 - protein, 6 aa, Artificial Sequence
  NCR3.24 LC3
TSSLPI

SEQ ID NO: 209 - protein, 10 aa, Artificial Sequence
  NCR3.24 HC3
WYMYYYKIAM

SEQ ID NO: 210 - protein, 10 aa, Artificial Sequence
  NCR3.25 HC2
SISSYYGSTS

SEQ ID NO: 211 - protein, 18 aa, Artificial Sequence
  NCR3.25 HC3
QWEYSSYHYSTWYYAWAM

SEQ ID NO: 212 - protein, 18 aa, Artificial Sequence
  NCR3.26 HC3
RYDSYYWYYYSSYWYYGM

SEQ ID NO: 213 - protein, 6 aa, Artificial Sequence
  NCR3.27 LC3
LYSRLV

SEQ ID NO: 214 - protein, 12 aa, Artificial Sequence
  NCR3.27 HC3
PYDYYGWYWGAF

SEQ ID NO: 215 - protein, 8 aa, Artificial Sequence
  TNFRSF9.01 LC3
GSWYSGLI

SEQ ID NO: 216 - protein, 10 aa, Artificial Sequence
  TNFRSF9.01 HC2
SIYCSYGSTY

SEQ ID NO: 217 - protein, 7 aa, Artificial Sequence
  TNFRSF9.01 HC3
NWYWYAL

SEQ ID NO: 218 - protein, 7 aa, Artificial Sequence
  TNFRSF9.02 LC3
SYYYDPV

SEQ ID NO: 219 - protein, 6 aa, Artificial Sequence
  TNFRSF9.02 HC1
IYSSSI

SEQ ID NO: 220 - protein, 10 aa, Artificial Sequence
  TNFRSF9.02 HC2
SIYSYSGSTY

SEQ ID NO: 221 - protein, 18 aa, Artificial Sequence
  TNFRSF9.02 HC3
EFVQWHYGYFYYDDWYAF

SEQ ID NO: 222 - protein, 7 aa, Artificial Sequence
  TNFRSF9.03 LC3
SSLGYLI

SEQ ID NO: 223 - protein, 10 aa, Artificial Sequence
  TNFRSF9.03 HC2
SISSYYGYTS

SEQ ID NO: 224 - protein, 8 aa, Artificial Sequence
  TNFRSF9.03 HC3
EWWEGWAL

SEQ ID NO: 225 - protein, 7 aa, Artificial Sequence
  TNFSF4.01 LC3
TSSSYLI

SEQ ID NO: 226 - protein, 10 aa, Artificial Sequence
  TNFSF4.01 HC2
SIYPYSGSTS

SEQ ID NO: 227 - protein, 12 aa, Artificial Sequence
  TNFSF4.01 HC3
WYQYSRPRDWGL

SEQ ID NO: 228 - protein, 8 aa, Artificial Sequence
  TNFSF4.02 LC3
GYSSSSLI

SEQ ID NO: 229 - protein, 6 aa, Artificial Sequence
  TNFSF4.02 HC1
IYSYSI

SEQ ID NO: 230 - protein, 10 aa, Artificial Sequence
  TNFSF4.02 HC2
SIYPYYSYTS

SEQ ID NO: 231 - protein, 10 aa, Artificial Sequence
  TNFSF4.02 HC3
WYMYAEADAM

SEQ ID NO: 232 - protein, 7 aa, Artificial Sequence
  TNFSF4.04 LC3
SWVSGPF

SEQ ID NO: 233 - protein, 10 aa, Artificial Sequence
  TNFSF4.04 HC2
SIYSYSGYTS

SEQ ID NO: 234 - protein, 10 aa, Artificial Sequence
  TNFSF4.04 HC3
WYHHFYHMAM

SEQ ID NO: 235 - protein, 10 aa, Artificial Sequence
  TNFSF4.06 HC2
SISPLLWLYF

SEQ ID NO: 236 - protein, 8 aa, Artificial Sequence
  TNFSF4.08 LC3
SYSDSSLF

SEQ ID NO: 237 - protein, 10 aa, Artificial Sequence
  TNFSF4.08 HC2
SIYPSYGSTY

SEQ ID NO: 238 - protein, 12 aa, Artificial Sequence
  TNFSF4.08 HC3
WYGYSYYSHEAM

SEQ ID NO: 239 - protein, 5 aa, Artificial Sequence
  GFP.01 LC3
SWGLI

SEQ ID NO: 240 - protein, 9 aa, Artificial Sequence
  GFP.01 HC3
AGWVASSGM

SEQ ID NO: 241 - protein, 18 aa, Artificial Sequence
  NCR3.18.16 HC3
RTSSYYISYYDSFYYAGM

(19) United States
(12) Patent Application Publication
(10) Pub. No.: US 2024/0279331 A1
(43) Pub. Date: Aug. 22, 2024

(54) ANTIBODIES THAT STIMULATE NK CELL-MEDIATED CYTOTOXICITY

(71) Applicant: The Regents of the University of California, Oakland, CA (US)

(72) Inventors: James A. Wells, San Francisco, CA (US); Emily Kang, San Francisco, CA (US); Lewis L. Lanier, San Francisco, CA (US)

(73) Assignee: The Regents of the University of California, Oakland, CA (US)

(21) Appl. No.: 18/568,218

(22) PCT Filed: Jun. 9, 2022
(86) PCT No.: PCT/US2022/032855

§ 371 Date: Dec. 7, 2023

Related U.S. Application Data
(60) Provisional application No. 63/209,671, filed on Jun. 11, 2021.

(51) Int. Cl. C07K 16/28; C07K 16/32

(52) U.S. Cl. CPC C07K 16/2803; C07K 16/2887; C07K 16/32; C07K 2317/31; C07K 2317/55; C07K 2317/622; C07K 2317/732; C07K 2317/92

(57) ABSTRACT

A functional screen to rapidly identify antibodies that can activate NK cells is provided as well as antibodies identified in the screen and their use.

### CROSS-REFERENCE TO RELATED PATENT APPLICATIONS

The present patent application claims benefit of priority to U.S. Provisional Patent Application No. 63/209,671, filed Jun. 11, 2021, which is incorporated by reference for all purposes.

### STATEMENT AS TO RIGHTS TO INVENTIONS MADE UNDER FEDERALLY SPONSORED RESEARCH AND DEVELOPMENT

This invention was made with government support under grant no. R35 GM122451 awarded by The National Institutes of Health. The government has certain rights in the invention.

### BACKGROUND OF THE INVENTION

Cancer immunotherapies have garnered a great deal of success over the last decade. Much of this success has been driven by the development of antibody-based therapeutics that redirect and enhance the cytotoxic potential of CD8+ T cells via immune checkpoint blockade or CD3/T cell receptor (TCR) complex stimulation. Like CD8+ T cells, natural killer (NK) cells are cytotoxic effector cells that mediate anti-tumor responses [Waldhauer, I. & Steinle, A., Oncogene 27(45), 5932-5943 (2008); Raulet, D. H. & Guerra, N., Nat. Rev. Immunol 9(8), 568-580 (2009); Marcus, A. et al., Adv. Immunol. 122, 91-128 (2014)]. They play a key role in tumor immunosurveillance and are able to identify and remove target cells by recognizing stress-induced ligands that are frequently overexpressed on cancer cells. NK cells are also known to perform antibody-dependent cellular cytotoxicity (ADCC), a mechanism that is used by multiple, current therapeutic monoclonal antibodies to eradicate tumor cells [Weng, W. K. & Levy, R., J Clin Oncol. 21(21), 3940-3947 (2003); Musolino, A. et al., J Clin Oncol. 26(11), 1789-1796 (2008); Rodriguez, J. et al., Eur. J. Cancer. 48(12), 1774-1780 (2012)]. Given the crucial role that NK cells play in tumor immunosurveillance, the identification of novel immunotherapies that can target and redirect NK cell cytotoxicity merits further investigation.

Whereas all T cells express the CD3/TCR complex that can be exploited by immunomodulatory molecules to redirect T cell activity, NK cells express multiple activating, costimulatory, and inhibitory receptors that govern NK cell activity [Lanier, L. L., Nat. Immunol. 9(5), 495-502 (2008); Chester, C., Fritsch, K., Kohrt, H. E., Front Immunol. 6, 601 (2015)]. Moreover, the NK cell repertoire is highly diverse and the expression of these activating and inhibitory receptors among different cell subsets varies greatly within and among individuals [Horowitz, A. et al., Sci. Transl. Med. 5(208), 208ra145 (2013); Strauss-Albee, D. M. et al., Sci. Transl. Med. 7(297), 297ra115 (2015)]. These factors make it difficult to develop antibodies that can recruit and stimulate NK cells.

### BRIEF SUMMARY OF THE INVENTION

In some embodiments, the disclosure provides an antibody that specifically binds to human Natural Cytotoxicity Triggering Receptor 3 (NCR3), wherein the antibody comprises at least:
(1) a light chain variable region comprising a light chain complementarity determining region (LCDR) 1 comprising SEQ ID NO:2, a LCDR2 comprising SEQ ID NO:3 and a LCDR3 comprising SEQ ID NO:4; and
a heavy chain variable region comprising a heavy chain complementarity determining region (HCDR) 1 comprising SEQ ID NO: 6, a HCDR2 comprising SEQ ID NO:7 and a HCDR3 comprising SEQ ID NO:8; or
(2) a light chain variable region comprising a light chain complementarity determining region (LCDR) 1 comprising SEQ ID NO: 10, a LCDR2 comprising SEQ ID NO:11 and a LCDR3 comprising SEQ ID NO: 12; and
a heavy chain variable region comprising a heavy chain complementarity determining region (HCDR) 1 comprising SEQ ID NO: 14, a HCDR2 comprising SEQ ID NO: 15 and a HCDR3 comprising SEQ ID NO:41; or
(3) a light chain variable region comprising a light chain complementarity determining region (LCDR) 1 comprising SEQ ID NO: 18, a LCDR2 comprising SEQ ID NO:19 and a LCDR3 comprising SEQ ID NO:20; and
a heavy chain variable region comprising a heavy chain complementarity determining region (HCDR) 1 comprising SEQ ID NO: 22, a HCDR2 comprising SEQ ID NO:23 and a HCDR3 comprising SEQ ID NO:24.

In some embodiments, the light chain variable region comprises a light chain complementarity determining region (LCDR) 1 comprising SEQ ID NO:10, a LCDR2 comprising SEQ ID NO:11 and a LCDR3 comprising SEQ ID NO:12; and the heavy chain variable region comprises a heavy chain complementarity determining region (HCDR) 1 comprising SEQ ID NO: 14, a HCDR2 comprising SEQ ID NO:15 and a HCDR3 comprising SEQ ID NO:41.

In some embodiments, the HCDR3 comprises one of SEQ ID NO:16, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, or SEQ ID NO:57.

In some embodiments, the light chain variable region comprises SEQ ID NO:1; and the light chain variable region comprises SEQ ID NO:5.

In some embodiments, the light chain variable region comprises SEQ ID NO:9; and the light chain variable region comprises SEQ ID NO:13.

In some embodiments, the light chain variable region comprises SEQ ID NO:17; and the light chain variable region comprises SEQ ID NO:21.

In some embodiments, the antibody is a bi-specific antibody that binds NCR3 and a second target protein. In some embodiments, the second target protein is expressed on cancer cells. In some embodiments, the second target protein is CD20 or BCMA or HER2.

Also provided is a polynucleotide encoding the antibody as described above.

Also provided is a cell that expresses the antibody as described above. In some embodiments, the cell is a mammalian cell.

Also provided is a method of stimulating natural killer (NK) cell-mediated cytotoxicity in a human in need thereof. In some embodiments, the method comprises administering the antibody as described above to the human in an amount sufficient to stimulate NK cell-mediated cytotoxicity. In some embodiments, the human has cancer and the NK cell-mediated cytotoxicity kills cancer cells. In some embodiments, the cancer is multiple myeloma, leukemia, Hodgkin's lymphoma or non-Hodgkin's lymphoma.

Also provided is an antibody that specifically binds to human Natural Cytotoxicity Triggering Receptor 1 (NCR1), wherein the antibody comprises at least a light chain variable region comprising a light chain complementarity determining region (LCDR) 1 comprising SEQ ID NO: 26, a LCDR2 comprising SEQ ID NO:27 and a LCDR3 comprising SEQ ID NO:28; and a heavy chain variable region comprising a heavy chain complementarity determining region (HCDR) 1 comprising SEQ ID NO: 30, a HCDR2 comprising SEQ ID NO:31 and a HCDR3 comprising SEQ ID NO: 32. In some embodiments, the light chain variable region comprises SEQ ID NO:25; and the light chain variable region comprises SEQ ID NO:29.

In some embodiments, the antibody is a bi-specific antibody that binds NCR1 and a second target protein. In some embodiments, the second target protein is expressed on cancer cells. In some embodiments, the second target protein is CD20 or BCMA or HER2.

Also provided is a polynucleotide encoding the antibody as described above.

Also provided is a cell that expresses the antibody as described above. In some embodiments, the cell is a mammalian cell.

Also provided is a method of stimulating natural killer (NK) cell-mediated cytotoxicity in a human in need thereof, the method comprising administering the antibody as described above to the human in an amount sufficient to stimulate NK cell-mediated cytotoxicity.

In some embodiments, the human has cancer and the NK cell-mediated cytotoxicity kills cancer cells. In some embodiments, the cancer is multiple myeloma, leukemia, Hodgkin's lymphoma or non-Hodgkin's lymphoma.

Also provided is an antibody that specifically binds to human CD-16, wherein the antibody comprises at least a light chain variable region comprising a light chain complementarity determining region (LCDR) 1 comprising SEQ ID NO: 34, a LCDR2 comprising SEQ ID NO:35 and a LCDR3 comprising SEQ ID NO:36; and a heavy chain variable region comprising a heavy chain complementarity determining region (HCDR) 1 comprising SEQ ID NO: 38, a HCDR2 comprising SEQ ID NO:39 and a HCDR3 comprising SEQ ID NO:40.

In some embodiments, the light chain variable region comprises SEQ ID NO:25; and the light chain variable region comprises SEQ ID NO:29.

In some embodiments, the antibody is a bi-specific antibody that binds CD-16 and a second target protein. In some embodiments, the second target protein is expressed on cancer cells. In some embodiments, the second target protein is CD20 or BCMA or HER2.

Also provided is a polynucleotide encoding the antibody as described above.

Also provided is a cell that expresses the antibody as described above. In some embodiments, the cell is a mammalian cell.

Also provided is a method of stimulating natural killer (NK) cell-mediated cytotoxicity in a human in need thereof, the method comprising administering the antibody as described above to the human in an amount sufficient to stimulate NK cell-mediated cytotoxicity. In some embodiments, the human has cancer and the NK cell-mediated cytotoxicity kills cancer cells. In some embodiments, the cancer is multiple myeloma, leukemia, Hodgkin's lymphoma or non-Hodgkin's lymphoma.

Also provided is a method of identifying antibodies that activate natural killer (NK) cells. In some embodiments, the method comprises, providing a library of antibodies that bind to proteins on NK cells; expressing the library of antibodies on the surface of mammalian cells; incubating a population of the mammalian cells with NK cells under conditions in which the NK cells kill at least some mammalian cells based on the antibody expressed on the cells; and following the incubating, quantifying the proportion of cells remaining; comparing the proportion of cells remaining to a control population of mammalian cells, wherein a decrease in the proportion of cells expressing a particular antibody indicates the particular antibody activates NK cells.

In some embodiments, the method further comprises contacting the particular antibody to an NK cell and measuring activation of the contacted NK cell. In some embodiments, the protein is selected from the group consisting of Natural Cytotoxicity Triggering Receptor 1 (NCR1), NCR3, and CD-16.

### BRIEF DESCRIPTION OF THE DRAWINGS

FIG. 1a-b: Schematic of functional screen. (a) Phage from NK cell antigen selections were screened via ELISA, and Fabs with unique CDRs were converted into scFabs. Jurkat cells were transduced with membrane bound (MB) scFabs to generate the mammalian display library. The library was incubated in the presence or absence of peripheral blood NK cells, and surviving cells were subjected to next-generation DNA sequencing to identify scFabs that were depleted by NK cells. (b) The scFab gene was integrated into the Jurkat genome. Sequencing of CDR H3 was used to distinguish different scFabs.

FIG. 2: Functional mammalian display screen identifies antibodies that stimulate NK cytotoxicity. 69 scFabs targeting 6 NK cell antigens were displayed on Jurkat cells to generate the mammalian display library. The library was incubated with resting or IL2 stimulated peripheral blood NK cells from two different donors for either 4 or 24 hours. NGS counts were normalized to the mammalian display library that were cultured for 4 hours or 24 hours in the absence of NK cells. Only four scFabs, CD16.03, NCR1.11, NCR3.18 and NCR3.19 were depleted by NK cells. Positive NGS signals (enriched) are shown in red, and negative NGS signals (depleted) are shown in blue.

FIG. 3a-b: In vitro activity of antibodies identified from functional screen against FcγR+ cell lines. (a) Redirected lysis assay using peripheral blood NK cells against FcγR+ THP-1 cells in the presence of varying concentrations of antibodies. Data are representative of 3 independent experiments. (b) IFN-γ secretion assay using peripheral blood NK cells against FcγR+ P815 cells in the presence or absence of varying concentrations of antibodies. All four antibodies that were identified as activating in the functional screen were able to elicit NK cytotoxicity and IFN-γ secretion. Only one antibody that was identified as non-stimulatory in the functional screen was able to elicit NK cytotoxicity and IFN-γ secretion. Values represent mean±SEM of 8 different donors. ***p<0.001. ****p<0.0001

FIG. 4a-f. Bispecific constructs generated and cytotoxicity induced by bispecific constructs against CD20+ Daudi. (a) Each NK targeting antibody was converted into an scFv (in blue) and attached to either the light chain or heavy chain of the tumor targeting Fab (in grey). The tumor antigen was either CD20 or HER2. Cytotoxicity induced by bispecific constructs against CD20+ Daudi. (b) Cytotoxicity induced by PBMCs in the presence of anti-CD20-scFv CD16.03 bispecifics at an E:T of 10:1. (c) Cytotoxicity induced by NCR1+ NKL cells in the presence of anti-CD20-scFv NCR1.11 bispecifics at an E:T of 3:1. (d) Cytotoxicity induced by NCR3+ NK92MI cells in the presence of anti-CD20-scFv NCR3.12 bispecifics at an E:T of 1:9. (e) Cytotoxicity induced by NCR3+NK92MI cells in the presence of anti-CD20-scFv NCR3.19 bispecifics at an E:T of 1:9. (f) Comparison of cytotoxicity induced by PBMCs to anti-CD20 human IgG1 mAb.

FIG. 5a-e. Cytotoxicity of SC1 lymphoma cells by bispecifics. (a) anti-CD20 Fab. (b) anti-CD20 human IgG1 mAb. (c) anti-CD20-scFv CD16.03 bispecifics. (d) anti-CD20-scFv NCR1.11 bispecifics. (e) anti-CD20-scFv NCR3.12 bispecifics.

FIG. 6 (S1). Schematic of phage display selections and Fc-fusion construct used to enrich for Fab phage that selectively bind to NK cell antigens.

FIG. 7 (S2). Fab-phage ELISAs from selections against NK cell antigens to identify high affinity binders. On the y-axis, direct binding of Fab-phage to the antigen of interest is plotted. On the x-axis, the competition to direct ratio is shown. For competitive binding, Fab-phage is preincubated with 20 nM of soluble antigen and then allowed to bind to the antigen coated plates

FIG. 8a-b (S3). Expression of membrane bound (MB) scFab library on mammalian target cells. (a) Format of construct used to display scFabs on mammalian target cells. (b) Expression of MB scFab library on Jurkat target cells. WT Jurkats are in red. MB-scFAB expressing Jurkats are in blue. Expression of scFab at the cell surface was detected with an anti-human Fab antibody.

FIG. 9 (S4). Comparisons of biological replicates from two different blood donors correlate well.

FIG. 10 (S5). Representative flow cytometry histograms demonstrate the selectivity of resultant Fabs towards the antigen they were selected against. Six tetracycline-inducible FlpIn cell lines were generated to over-express each NK cell antigens upon tetracycline addition.

FIG. 11 (S6). Titration of antibodies identified from functional screen against peripheral blood NK cells. Median fluorescence intensities of NK cells were plotted over a range of antibody concentrations. Antibodies that were identified as activating from the functional screen bound at lower concentrations than almost all of the antibodies that were identified as non-functional.

FIG. 12a-d (S7). Titration of bispecific antibodies towards HEK293 FlpIn cell lines over expressing NK antigens of interest. (a) Binding of anti-CD20-scFv CD16.03 bispecifics towards CD16 over expression cell line. (b) Binding of anti-CD20-scFv NCR1.11 bispecifics towards NCR1 over expression cell line. (c) Binding of anti-CD20-scFv NCR3.12 bispecifics towards NCR3 over expression cell line. (d) Binding of anti-CD20-scFv NCR3.19 bispecifics towards NCR3 over expression cell line.

FIG. 13 (S8) Cytotoxicity induced by HER2 targeting bispecific constructs.

FIG. 14a-b (S9) CD20 expression levels on SC1 lymphoma cells and on CD20+ Daudi cells. (a) SC1 lymphoma cells or (b) CD20+ Daudi cells were incubated with 10, 1, 0.1, or 0 μg/mL of biotinylated anti-CD20 IgG1 mAb and subsequently stained with a streptavidin-AlexaFluor-647 secondary to determine CD20 expression levels.

### DEFINITIONS

As used in herein, the singular forms “a”, “an” and “the” include plural referents unless the content clearly dictates otherwise. Thus, for example, reference to “an antibody” optionally includes a combination of two or more such molecules, and the like.

As used herein, the term “antibody” means an isolated or recombinant binding agent that comprises the necessary variable region sequences to specifically bind an antigenic epitope. Therefore, an “antibody” as used herein is any form of antibody of any class or subclass or fragment thereof that exhibits the desired biological activity, e.g., binding a specific target antigen. Thus, it is used in the broadest sense and includes, but is not limited to, a monoclonal antibody (including full-length monoclonal antibodies), human antibodies, chimeric antibodies, single domain antibodies, such as nanobodies, diabodies, camelid-derived antibodies, monovalent antibodies, bivalent antibodies, multivalent antibodies, multispecific antibodies (e.g., bispecific antibodies), and antibody fragments including, but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)₂, and Fv fragments; diabodies; linear antibodies; single-chain antibody molecules (e.g., scFv); and multispecific or multivalent antibodies formed from antibody fragments. A “Fab” fragment contains a variable and constant domain of the light chain and a variable domain and the first constant domain (CH1) of the heavy chain. A F(ab′)₂ fragment has a pair of Fab fragments that are generally covalently linked near their carboxy termini by hinge cysteines. Other chemical couplings of antibody fragments are also known. An “Fv” is a minimal antibody fragment that contains a complete antigen-recognition and binding site and is a dimer of one heavy- and one light-chain variable region domain.

The “class” of an antibody refers to the type of constant domain or constant region possessed by its heavy chain. There are five major classes of antibodies: IgA, IgD, IgE, IgG, and IgM, and may be further divided into subclasses (isotypes), e.g., IgG₁, IgG₂, IgG₃, IgG₄. The antibodies described herein can be of any of these classes or subclasses.

As used herein, “V-region” refers to an antibody variable region domain comprising the segments of Framework 1, CDR1, Framework 2, CDR2, and Framework 3, including CDR3 and Framework 4.

As used herein, “complementarity-determining region (CDR)” refers to the three hypervariable regions that interrupt the four “framework” regions of s variable domain. The CDRs are the primary contributors to binding to an epitope of an antigen. The CDRs of each heavy or light chain are referred to as CDR1, CDR2, and CDR3, numbered sequentially starting from the N-terminus.

The amino acid sequences of the CDRs and framework regions can be determined using various well-known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883, Chothia C. et al., 1992, structural repertoire of the human VH segments J. Mol. Biol. 227, 799-817; Al-Lazikani et al., J. Mol. Biol 1997, 273(4)). Definitions of CDRs are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 19%). Reference to CDRs as determined by Kabat numbering are based, for example, on Kabat et al, Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)).

“Epitope” or “antigenic determinant” as used in the present disclosure in the context of antibody binding refers to a site on an antigen to which an antibody binds. Epitopes can be formed from contiguous amino acids and/or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). Binding of an antibody to an epitope can be influenced by other environmental factors, such as s the presence of calcium ions.

The term “valency” as used herein refers to the number of different binding sites of an antibody for an antigen. A monovalent antibody comprises one binding site for an antigen. A multivalent antibody comprises multiple binding sites.

The term “monovalent antibody” as used herein, refers to an antibody that binds to a single epitope on a target molecule.

The term “bivalent antibody” as used herein, refers to an antibody that has two antigen binding sites.

The term “multivalent antibody” refers to a single binding molecule with more than one valency, where “valency” is described as the number of antigen-binding moieties present per molecule of an antibody construct. As such, the single binding molecule can bind to more than one binding site on a target molecule. Examples of multivalent antibodies include, but are not limited to, bivalent antibodies, trivalent antibodies, tetravalent antibodies, pentavalent antibodies, and the like, as well as bispecific antibodies.

The term “bispecific antibody” as used herein, refers to an antibody that binds to two or more different epitopes. In some embodiments, a bispecific antibody binds to epitopes for two different target antigens. In some embodiments, a bispecific antibody binds to two different epitopes for the same target antigen. Bi-specific antibodies can be made in several ways. In some embodiments, the bi-specific antibodies described herein are knob-in-a-hole IgG antibodies or otherwise use knob-in-a-hole technology. See, e.g., Xu, et al., MAbs 7(1):231-42 (2015).

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

As used herein, the term “specifically binds” to a target, e.g., NCR1, NCR3 or CD-16, refers to a binding reaction whereby the antibody binds to the target with greater affinity, greater avidity, and/or greater duration than it binds to a different target. In some embodiments, a target-binding protein has at least 2-fold, 3-fold, 4-fold, 5-fold, 6-fold, 7-fold, 8-fold, 9-fold, 10-fold, 20-fold, 25-fold, 50-fold, 100-fold, 1,000-fold, 10,000-fold, or greater affinity for the target compared to an unrelated target when assayed under the same binding affinity assay conditions. The term “specific binding,” “specifically binds to,” or “is specific for” a particular target, as used herein, can be exhibited, for example, by a molecule (e.g., an antibody) having an equilibrium dissociation constant K_{D} for the target of, e.g., 10⁻² M or smaller, e.g., 10⁻³ M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some embodiments, an antibody has a K_{D} of less than 100 nM or less than 10 nM.

The term “treat” and “treatment” refer to both therapeutic treatment and prophylactic or preventive measures, wherein the object is to prevent or slow down an undesired physiological change or disorder. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

As used herein, the term “subject” refers to a mammal, e.g., preferably a human. Mammals include, but are not limited to, humans and domestic and farm animals, such as monkeys (e.g., a cynomolgus monkey), mice, dogs, cats, horses, and cows, etc.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. In the present invention, the pharmaceutically acceptable carrier must provide adequate pharmaceutical stability to the active ingredient. The nature of the carrier differs with the mode of administration. For example, for intravenous administration, an aqueous solution carrier is generally used; for oral administration, a solid carrier is preferred.

### DETAILED DESCRIPTION OF THE INVENTION

The inventors have discovered novel antibodies that bind to and activate NK cells as well as methods for identifying novel reagents that activate NK cells. A functional screen to rapidly identify antibodies that can activate NK cells was developed. Antibodies were displayed on a mammalian target cell line and probed their ability to stimulate NK cell-mediated cytotoxicity. From this screen, antibodies specific for NCR1, NCR3 and CD-16 were identified that bound with high affinity to NK cells and subsequent-developed bispecific antibody constructs were shown to redirect NK cell-mediated cytotoxicity towards CD20+ B cell lymphomas. Thus by targeting (for example but not limited to via a bi-specific antibody) the antibodies described herein to a cell of interest, NK cells can be targeted to the cell to kill it.

Exemplary antibodies described herein include those that specifically bind to NCR1, NCR3 and CD-16. Not all antibodies that bind to these targets activate NK cells but those antibodies described herein do as demonstrated in the examples.

Exemplary anti-NCR1 antibodies described herein include those having a light chain variable region comprising LCDR1 comprising RASQSVSSAV (SEQ ID NO:26), LCDR2 comprising SASSLYS (SEQ ID NO:27) and LCDR3 SSSSLI (SEQ ID NO:28) and a heavy chain variable region comprising HCDR1 comprising VYYSYT (SEQ ID NO:30), HCDR2 comprising SISSYYGSTY (SEQ ID NO:31), and comprising HCDR3 SRYLQDYWSSWWVSWYGL (SEQ ID NO:32). In some embodiments the light chain variable region comprises SEQ ID NO:25 (optionally with 1, 2 or 3 amino acid changes, which may be conservative amino acid changes), the heavy chain variable region comprises SEQ ID NO:29 (optionally with 1, 2 or 3 amino acid changes, which may be conservative amino acid changes), or both.

Exemplary anti-NCR3 antibodies described herein include:

(1) Those having a light chain variable region comprising LCDR1 comprising RASQSVSSAV (SEQ ID NO:2), LCDR2 comprising SASSLYS (SEQ ID NO:3) and LCDR3 SSYWPF (SEQ ID NO:4) and a heavy chain variable region comprising HCDR1 comprising ISSSSI (SEQ ID NO:6), HCDR2 comprising YISSSSGYTS (SEQ ID NO:7), and comprising HCDR3 YSYFYGGYFYWTSWGAF (SEQ ID NO:8). In some embodiments the light chain variable region comprises SEQ ID NO:1 (optionally with 1, 2 or 3 amino acid changes, which may be conservative amino acid changes), the heavy chain variable region comprises SEQ ID NO:5 (optionally with 1, 2 or 3 amino acid changes, which may be conservative amino acid changes), or both.

(2) Those having a light chain variable region comprising LCDR1 comprising RASQSVSSAV (SEQ ID NO:10), LCDR2 comprising SASSLYS (SEQ ID NO:11) and LCDR3 SSSSLI (SEQ ID NO:12) and a heavy chain variable region comprising HCDR1 comprising VSSSSI (SEQ ID NO: 14), HCDR2 comprising STSSSSGSTS (SEQ TD NO:15), and comprising HCDR3 RX(G/A/S)SX(Y/F)X(S/T)YYDSFYYAG(M/L) where X is any amino acid. In some embodiments, the HCDR3 comprises one of RISSYYMSYYDSFYYAGM (SEQ ID NO:16); RASSRFRSYYDSFYYAGM (SEQ ID NO:42); RIGSIYRSYYDSFYYAGM (SEQ ID NO:43); RISSHYMSYYDSFYYAGM (SEQ ID NO:44); RISSSYMSYYDSFYYAGM (SEQ ID NO:45); RISSYYISYYDSFYYAGM (SEQ ID NO:46); RISSYYVSYYDSFYYAGM (SEQ ID NO:47); RKSSSYWSYYDSFYYAGM (SEQ ID NO:48); RKSSYYMSYYDSFYYAGM (SEQ ID NO:49); RLGSRYRSYYDSFYYAGM (SEQ ID NO:50); RRASYYKTYYDSFYYAGM (SEQ ID NO:51); RRSSYYMTYYDSFYYAGM (SEQ ID NO:52); RTGSYYMTYYDSFYYAGM (SEQ ID NO:53); RTSSHYISYYDSFYYAGM (SEQ ID NO:54). RVGSYYMSYYDSFYYAGM (SEQ ID NO:55); RVSSNYMSYYDSFYYAGM (SEQ ID NO:56); or RVSSPYMSYYDSFYYAGL (SEQ ID NO:57). In some embodiments the light chain variable region comprises SEQ ID NO:9 (optionally with 1, 2 or 3 amino acid changes, which may be conservative amino acid changes), the heavy chain variable region comprises SEQ ID NO:13 (optionally with 1, 2 or 3 amino acid changes, which may be conservative amino acid changes), or both.

(3) Those having a light chain variable region comprising LCDR1 comprising RASQSVSSAV (SEQ ID NO:18), LCDR2 comprising SASSLYS (SEQ ID NO:19) and LCDR3 QWYPLI (SEQ ID NO:20) and a heavy chain variable region comprising HCDR1 comprising VYSYSI (SEQ ID NO:22), HCDR2 comprising SIYSYYGSTS (SEQ ID NO:23), and comprising HCDR3 WYQYYYIGTAAM (SEQ ID NO:24). In some embodiments the light chain variable region comprises SEQ ID NO: 17 (optionally with 1, 2 or 3 amino acid changes, which may be conservative amino acid changes), the heavy chain variable region comprises SEQ ID NO:21 (optionally with 1, 2 or 3 amino acid changes, which may be conservative amino acid changes), or both.

Exemplary anti-NCR1 antibodies described herein include those having a light chain variable region comprising LCDR1 comprising RASQSVSSAV (SEQ ID NO:34), LCDR2 comprising SASSLYS (SEQ ID NO:35) and LCDR3 SSAELI (SEQ ID NO:36) and a heavy chain variable region comprising HCDR1 comprising FSSYSI (SEQ ID NO:38), HCDR2 comprising SIYSSSGSTS (SEQ ID NO:39), and comprising HCDR3 WSYDQYYDQHGYYFYYWGF (SEQ TD NO:40) In some embodiments the light chain variable region comprises SEQ TD NO:33 (optionally with 1, 2 or 3 amino acid changes, which may be conservative amino acid changes), the heavy chain variable region comprises SEQ ID NO:37 (optionally with 1, 2 or 3 amino acid changes, which may be conservative amino acid changes), or both.

In view of the NK-activating activity of the antibodies described herein, linking or tagging the antibodies described herein to a target cell will result in NK cells attacking and killing that target cell. The antibodies described herein can be linked or tagged to a target cells in any way desired. In some embodiments, the heavy and/or light chain variable regions that target the NK proteins (NCR1, NCR3, or CD-16) are fused to a separate amino acid sequence(s) that targets the resulting fusion protein to the target cell. In these embodiments, the fusion protein is contacted to the surface of the target cell. As one example, as described more fully below, bi-specific antibodies that comprise an NK cell-binding domain (e.g., NCR1, NCR3, or CD-16 binding domain) as well as a binding domain that targets the target cell can be used.

Exemplary binding domains can be for example heavy and light chain variable sequences comprising at least the CDRs described herein. Illustrative antibody and antibody fragment formats are described in detail in Brinkmann et al. (MABS, 2017, Vol. 9, No. 2, 182-212). Thus in some embodiments, a NCR1, NCR3, or CD-16-binding domain, e.g., a VH region and/or a VL region of an antibody as described herein, may be incorporated into a bivalent antibody or a multivalent antibody that also binds to a different, antigen (e.g., a specific protein or other antigen on the target cell as described above). For example in some embodiments a multi-valent (e.g., bi-valent) antibody is provided that binds to NCR1, NCR3 or CD-16 and also binds to an epitope on a target cell, allowing for bringing the target cell in proximity to the antibody, which also binds to and stimulates an NK cell. While bi-specific antibodies are one way one can target the NK cell-binding domain to a different target cell, any type of affinity agent for the target cell can be linked or fused to the NK cell-binding domains described herein.

In some embodiments, as demonstrated in the examples, a NK cell-binding domain as described herein can be expressed in a target cell, resulting in killing of the cell expressing the NK binding domain. In some embodiments, the cell expressing the NK cell-binding domain can express the NK cell-binding domain only under particular conditions, for example under the control of an inducible promoter. Thus, such cells can be conditionally targeted for killing only upon induction of the NK cell-binding domain. Any cell that might be introduced into an animal can be designed in this way. For example, in some embodiments a cell-based therapy can be ended after a desired effect of the cell therapy by inducing expression of the NK cell-binding domain.

In any of the embodiments described herein, the NK cell-binding domain is targeted to a target cell such that NK cells kill the target cell. Any undesired cell can be the target cell. Exemplary target cells can include but are not limited to cancer cells. Exemplary cancer cells can include, but are not limited to myeloma, lymphoma and leukemia. In some embodiments, the NK cell-binding domain is targeted to specific protein or other antigen expressed on the surface of the target cell. In some embodiments, the specific protein or other antigen expressed on the target cell is specifically expressed or primarily expressed on the target cell compared to other cells in an animal (e.g., a human under treatment). This will reduce potential undesirable off-target cell killing. Exemplary proteins that can be targeted on cancer cells can include, but are not limited to CD19, CD20, CD22, CD33, CD30, CDCP1, EpCAM, GD2, HER2, BCMA, EGFR, PDGFRa, SLAMF7. See, also, world wide web at actip.org/products/monoclonal-antibodies-approved-by-the-ema-and-fda-for-therapeutic-use/, which describes monoclonal antibodies approved by the EMA and FDA for therapeutic use as of 2017 and their targets, of which those on the surface of the cell can be used in the method and compositions described herein. Other cancer antigens are known and can also be used. Exemplary further cancer antigens are described in, e.g., PCT/US2017/045632. Exemplary antibodies that bind to CD20 and whose variable regions can be used to generate bi-specific antibodies as described herein are known, e.g., in patents EP0605442; EP0669836; U.S. Pat. Nos. 7,381,560; 8,529,902; and 8,206,711. Exemplary antibodies that bind to HER2 and whose variable regions can be used to generate bi-specific antibodies as described herein are known, e.g., in patents EP0590058, U.S. Pat. Nos. 8,937,159; 9,862,769; 5,677,171. Exemplary antibodies that bind to BCMA and whose variable regions can be used to generate bi-specific antibodies as described herein are known, e.g., GSK2857916 (Belantamab Mafodotin); Tai Y. T., et al. Blood 2014; 123:3128-3138.

In some embodiments, the antibody comprising a NCR1, NCR3 or CD-16-binding domain as described herein further comprises an Fc region. The term “Fc region” as used herein refers to a polypeptide comprising the CH3, CH2 and at least a portion of the hinge region of a constant domain of an antibody. In some embodiments, an Fc region can include a CH4 domain, present in some antibody classes. In some embodiments, an Fc region, can comprise the entire hinge region of a constant domain of an antibody. In one embodiment, an antibody comprises an Fc region and a CH1 region. In one embodiment, the antibody comprises an Fc region, a CH1 region and a Ckappa/lambda region. In one embodiment, an antibody comprises a constant region, e.g., a heavy chain constant region. In some embodiments, such a constant region is modified compared to a wild-type constant region. i.e., a constant region may comprise alterations or modifications to one or more of the CH1, CH2 or CH3 domain and/or to the CL domain. Example modifications include additions, deletions or substitutions of one or more amino acids in one or more domains. Illustrative mutations are known, e.g., mutations that modulate effector function and/or serum half-life.

In some embodiments, a NCR1, NCR3 or CD-16-binding domain comprises an antibody fragment, e.g., a Fab, a F(ab′)₂, an Fv, an scFv antibody, a V_{H}, or a VHH. In some embodiments, a NCR1, NCR3 or CD-16-binding domain is provided in an scFV antibody as part of a bispecific antibody. Thus, for example, in some aspects, a NCR1, NCR3 or CD-16-binding domain can be incorporated into a bispecific antibody having a second binding domain that targets a different antigen on a non-NK cell, such as a cancer cell.

In some embodiments, an antibody as described herein (e.g., comprising a NCR1, NCR3 or CD-16-binding domain) may be a chimeric antibody, an affinity-mature, humanized, or human antibody.

Genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Optionally, phage or yeast display technology can be used to identify antibodies and Fab fragments that specifically bind to a target (e.g., NK-cell target protein) and/or other selected antigen of a bispecific antibody. Techniques for the production of single chain antibodies or recombinant antibodies can also be adapted to produce antibodies.

For example, the disclosure provides polynucleotides encoding a heavy chain variable region, light chain variable region or both as described herein. For example the polynucleotide can encode an antibody that specifically binds to human Natural Cytotoxicity Triggering Receptor 3 (NCR3), wherein the antibody comprises a light chain variable region comprising a light chain complementarity determining region (LCDR) 1 comprising SEQ ID NO:2, a LCDR2 comprising SEQ ID NO:3 and a LCDR3 comprising SEQ ID NO:4; and/or a heavy chain variable region comprising a heavy chain complementarity determining region (HCDR) 1 comprising SEQ ID NO: 6, a HCDR2 comprising SEQ ID NO:7 and a HCDR3 comprising SEQ ID NO:8. In some embodiments, the light chain variable region encoded by the polynucleotide comprises SEQ ID NO: 1; and/or the light chain variable region encoded by the polynucleotide comprises SEQ ID NO:5.

In some embodiments, the polynucleotide can encode an antibody that specifically binds to human Natural Cytotoxicity Triggering Receptor 3 (NCR3), wherein the antibody comprises a light chain variable region comprising a light chain complementarity determining region (LCDR) 1 comprising SEQ ID NO: 10, a LCDR2 comprising SEQ ID NO:11 and a LCDR3 comprising SEQ ID NO:12; and/or a heavy chain variable region comprising a heavy chain complementarity determining region (HCDR) 1 comprising SEQ ID NO: 14, a HCDR2 comprising SEQ ID NO: 15 and a HCDR3 comprising SEQ ID NO:41. In some embodiments, the HCDR3 comprises one of {#78 and new variants} SEQ ID NO:16, SEQ ID NO:42, SEQ ID NO:43, SEQ ID NO:4, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, or SEQ ID NO:57 In some embodiments, the light chain variable region encoded by the polynucleotide comprises SEQ ID NO:9; and/or the light chain variable region encoded by the polynucleotide comprises SEQ ID NO: 13.

In some embodiments, the polynucleotide can encode an antibody that specifically binds to human Natural Cytotoxicity Triggering Receptor 3 (NCR3), wherein the antibody comprises a light chain variable region comprising a light chain complementarity determining region (LCDR) 1 comprising SEQ ID NO: 18, a LCDR2 comprising SEQ ID NO:19 and a LCDR3 comprising SEQ ID NO:20; and/or a heavy chain variable region comprising a heavy chain complementarity determining region (HCDR) 1 comprising SEQ ID NO: 22, a HCDR2 comprising SEQ ID NO:23 and a HCDR3 comprising SEQ ID NO:24. In some embodiments, the light chain variable region encoded by the polynucleotide comprises SEQ ID NO:17; and/or the light chain variable region encoded by the polynucleotide comprises SEQ ID NO:21.

In some embodiments, the polynucleotide can encode an antibody that specifically binds to human Natural Cytotoxicity Triggering Receptor 1 (NCR1), wherein the antibody comprises at least a light chain variable region comprising a light chain complementarity determining region (LCDR) 1 comprising SEQ ID NO: 26, a LCDR2 comprising SEQ ID NO:27 and a LCDR3 comprising SEQ ID NO:28; and a heavy chain variable region comprising a heavy chain complementarity determining region (HCDR) 1 comprising SEQ ID NO: 30, a HCDR2 comprising SEQ ID NO:31 and a HCDR3 comprising SEQ ID NO:32. In some embodiments, the light chain variable region comprises SEQ ID NO:25; and the light chain variable region comprises SEQ ID NO:29.

In some embodiments, the polynucleotide can encode an antibody that specifically binds to human CD-16, wherein the antibody comprises at least a light chain variable region comprising a light chain complementarity determining region (LCDR) 1 comprising SEQ ID NO: 34, a LCDR2 comprising SEQ ID NO:35 and a LCDR3 comprising SEQ ID NO:36; and a heavy chain variable region comprising a heavy chain complementarity determining region (HCDR) 1 comprising SEQ ID NO: 38, a HCDR2 comprising SEQ ID NO:39 and a HCDR3 comprising SEQ ID NO:40. In some embodiments, the light chain variable region comprises SEQ ID NO:25; and the light chain variable region comprises SEQ ID NO:29.

Exemplary sequences encoding the above antibody sequences are shown in SEQ ID NOs:58-67, though it will be recognized that in view of the degeneracy of the genetic code other polynucleotide sequences can also encode the same amino acid sequence and are encompassed by the use of “polynucleotide.

Antibodies can be produced using any number of expression systems, including prokaryotic cell and eukaryotic cell expression systems. In some embodiments, the expression system is a mammalian cell expression, such as a hybridoma, or a CHO cell expression system. Many such systems are widely available from commercial suppliers. In embodiments in which an antibody comprises both a VH and VL region, the VH and VL regions may be expressed using a single vector, e.g., in a di-cistronic expression unit, or under the control of different promoters. In other embodiments, the VH and VL region may be expressed using separate vectors. A VH or VL region as described herein may optionally comprise a methionine at the N-terminus. Methods of generating and screening hybridoma cell lines, including the selection and immunization of suitable animals, the isolation and fusion of appropriate cells to create the hybridomas, the screening of hybridomas for the secretion of desired antibodies, and characterization of the antibodies are known to one of ordinary skill in the art.

In some embodiments, the antibody is a chimeric antibody. Methods for making chimeric antibodies are known in the art. For example, chimeric antibodies can be made in which the antigen-binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, the antibody is a humanized antibody. Generally, a non-human antibody is humanized in order to reduce its immunogenicity. Humanized antibodies typically comprise one or more variable regions (e.g., CDRs) or portions thereof that are non-human (e.g., derived from a mouse variable region sequence), and possibly some framework regions or portions thereof that are non-human, and further comprise one or more constant regions that are derived from human antibody sequences. Methods for humanizing non-human antibodies are known in the art. Transgenic mice, or other organisms such as other mammals, can be used to express humanized or human antibodies. Other methods of humanizing antibodies include, for example, variable region resurfacing, CDR grafting, grafting specificity-determining residues (SDR), guided selection, and framework shuffling.

Pharmaceutical compositions comprising an antibody as described herein can include one or more pharmaceutically acceptable carriers. Acceptable carriers and excipients in the pharmaceutical compositions are nontoxic to recipients at the dosages and concentrations employed. Acceptable carriers and excipients may include buffers, antioxidants, preservatives, polymers, amino acids, and carbohydrates. Pharmaceutical compositions may be administered parenterally in the form of an injectable formulation. Pharmaceutical compositions for injection (i.e., intravenous injection) can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2nd ed.) Taylor & Francis Group, CRC Press (2006).

The pharmaceutical composition may be formed in a unit dose form as needed. The amount of active component, e.g., an antibody as described herein, included in the pharmaceutical preparations is such that a suitable dose within the designated range is provided (e.g., a dose within the range of 0.01-500 mg/kg of body weight).

Pharmaceutical compositions described herein may be formulated for subcutaneous administration, intramuscular administration, intravenous administration, parenteral administration, intra-arterial administration, intrathecal administration, or intraperitoneal administration. The pharmaceutical composition may also be formulated for, or administered via, oral, nasal, spray, aerosol, rectal, or vaginal administration. For injectable formulations, various effective pharmaceutical carriers are known in the art. In some embodiments, pharmaceutical compositions may administered locally or systemically (e.g., locally). In particular embodiments, pharmaceutical compositions may be administered locally at the affected area, such as skin or cancerous tissue.

The dosage of the pharmaceutical compositions depends on factors including the route of administration, the disease to be treated, and physical characteristics, e.g., age, weight, general health, of the subject. In some embodiments, the amount of active ingredient (e.g., an antibody as described herein) contained within a single dose are administered in an amount that effectively prevents, delays, or treats the disease without inducing significant toxicity. The dosage may be adapted by the physician in accordance with conventional factors such as the extent of the disease and different parameters of the subject.

The pharmaceutical compositions may be administered in a manner compatible with the dosage formulation and in such amount as is therapeutically effective to result in an improvement or remediation of the symptoms. The pharmaceutical compositions may be administered in a variety of dosage forms, e.g., subcutaneous dosage forms, intravenous dosage forms, and oral dosage forms (e.g., ingestible solutions, drug release capsules). Pharmaceutical compositions containing the active ingredient (e.g., an anti-NK-cell protein target, e.g., an anti-NCR 1, NCR3, or CD-16 antibody) may be administered to a subject in need thereof, for example, one or more times (e.g., 1-10 times or more) daily, weekly, monthly, biannually, annually, or as medically necessary. Dosages may be provided in either a single or multiple dosage regimens. The timing between administrations may decrease as the medical condition improves or increase as the health of the patient declines.

The antibodies described herein (including binding fragments thereof, labeled antibodies, immunoconjugates, pharmaceutical compositions, etc.) can be used to induce NK-cell killing of target cells by targeting the antibodies to the target cell, thereby attracting and activating NK cells. In some embodiments, the antibodies can be used to treat, ameliorate, or prevent cancer as described herein. Accordingly, the antibodies and pharmaceutical compositions described herein can be administered to a human having or suspected of having cancer in an appropriate dosage to ameliorate or treat one of the cancer or at least one symptom thereof.

Also provided are methods for identifying agents that activate NK cells. For example, one can identify antibodies or other binding agents (e.g., aptamers, peptides, etc.) that activate NK cells by (i) expressing on cells binding agents (e.g., antibodies) that bind to known or potential NK-activating receptors, (ii) exposing the cells to NK cells, and (iii) determining the sequence of the individual binding agents (e.g., antibodies) on the cells that are killed, thereby identifying NK receptor activating antibodies. In some embodiments, the binding agents used will already have been selected for the ability to bind to a surface protein on NK cells, for example NCR1, NCR3 or CD-16 though this list should not be considered limiting. The binding agents can then be expressed on the surface of a cell. A plurality of binding agents can be considered a “library”, i.e., more than one different binding agent. In some embodiments, there are more than 5, 10, 20 or more binding agents tested. In some embodiments, a single binding agent can be assayed for activity. The cell used will not be attacked by NK cells unless an NK-cell activating agent is expressed on its surface. For example, the cells can be a mammalian cell, e.g., a human cell, e.g., Jurkat cells. The cells can then be exposed to NK cells under conditions and for a sufficient time such that cells that express an NK cell-activating binding agent are killed by the NK cells but other cells are not. By comparing the resulting cell population to a control population, e.g., one not contacted with NK cells, one can identify which cells were killed and thus which binding agents were able to activate the NK cells to kill the cells. In some embodiments, the identity of activating binding agents can be determined by performing nucleotide sequencing binding agents in the cells in the control cells compared to the NK-cell-treated cells and quantifying sequence reads for the binding agents. Any number of “next generation sequencing (NGS)” platforms can be used to perform, for example deep sequencing allowing for measurement of the quantity of sequencing reads representing particular binding agents expressed in the different cells. By comparing the proportion of cells remaining in the NK cell-treated cells to a control population of mammalian cells, one can identify binding agents associated with NK cell killing. Binding agents whose occurrences were reduced in the treated cells indicates the binding agents were able to active NK cells.

The following examples illustrate certain aspects of the claimed invention. It is understood that the examples and embodiments described herein are for illustrative purposes only and that various modifications or changes in light thereof will be suggested to persons skilled in the art and are to be included within the spirit and purview of this application and scope of the appended claims.

### Examples

We have developed a method to screen for antibodies that can induce NK cell-mediated cytotoxicity. NK cells have the innate ability to identify and kill target cells. Antibodies that bind to NK cell surface proteins are anchored to the cell surface of a target cell line and probed for their abilities to stimulate NK cytotoxicity. Target cells displaying antibodies that induce NK cell-mediated cytotoxicity are depleted from the antibody pool. Because the antibodies are based on the same scaffold, antibodies on surviving target cells can be identified through next generation sequencing (NGS) of complementarity determining region (CDR) H3. This method facilitates the identification of antibodies that can stimulate immune cell activation and may be used to design new immunotherapies.

We couple a mammalian display screen to a NGS readout to characterize antibodies that bind to and activate NK cells. Antibodies were selected against six NK cell receptors from a Fab-phage library that was based on the trastuzumab scaffold, and were displayed on a target cell line to generate a mammalian display library NK cells have the innate ability to recognize and kill unhealthy cells. We reasoned that an antibody against an NK cell surface protein that was displayed on a potential target cell could drive the interaction between an NK cell and a target cell. If the antibody were also able to activate NK cells, then the cell displaying the antibody would be killed and deselected. All of our antibodies are constructed on the same scaffold, allowing the use of the same set of primers to amplify and sequence the CDR H3 of each clone. Thus, we rationalized that we should be able to screen these antibodies in a pooled manner and quantify the depletion of specific antibody clones through NGS of CDR H3. Indeed, antibody binders that were depleted in our functional screen were able to stimulate NK cell cytotoxicity and IFN-γ secretion. We found that the most potent stimulators of NK cell-mediated cytotoxicity were high affinity binders to previously identified activating NK receptors, like CD16, NCR1, and NCR3, and that binding to other tested NK cell surface proteins was unable to stimulate NK cell activity. These activating antibodies were applied to the generation of bispecific antibodies to redirect NK cells towards CD20+ B cell lymphoma cells and HER2+ breast cancer cells. These results suggest that this method can facilitate the discovery of novel and rare antibodies that can stimulate immune cell activation and promote the design of new immunotherapies.

### Results

### Development of Antibodies Against NK Cells

In order to determine how to best target NK cells for the generation of NK cell-based immunotherapies, we sought to generate antibodies towards NK cell antigens with well-understood roles in NK cell activation. We chose to develop antibodies against CD16A [Mandelboim, O. et al., Proc Natl Acad Sci USA. 96(10), 5640-5644 (1999); Trinchieri, G. Valiante, N., Nat Immun. 12(4-5), 218-34 (1993)], NCR1 [Sivori, S. et al., J Exp Med. 186(7), 1129-36 (1997); Sivori, S et al., Eur J Immunol. 29(5), 1656-66 (199], and NCR3 [Pende, D. et al., J Exp Med. 190(10), 1505-16 (1991)], three well-characterized activating receptors that are known to initiate NK cell-mediated cytotoxicity. We also chose to generate antibodies against the costimulatory receptors, CD244 [Sivori, S. et al., Eur J Immunol. 30(3), 787-93 (2000)] and TNFRSF9 (4-1BB) [Srivastava, R. M. et al., Clin Cancer Res. 23(3), 707-716 (2017)], because costimulatory receptors can synergize with other activating receptors and signals [Bryceson, Y. T. et al., Blood. 107(1), 159-166 (2006); Bryceson, Y. T., Ljunggren, H. G. & Long, E. O., Blood. 114(13), 2657-2666 (2009)] to stimulate NK cells. Lastly, we chose to develop antibodies against TNFSF4 (OX40L), a ligand that can be upregulated upon NK stimulation [Zingoni, A., J Immunol. 173(6), 3716-3724 (2004)], but is not known to regulate NK cell-mediated cytotoxicity.

To generate antibodies against these antigens, we expressed the extracellular domains (ECDs) of these proteins as TEV-cleavable Fc-fusions and performed Fab-phage display selections to enrich for high affinity antibody binders (FIG. S1), as previously described [Hornsby, M. et al., Mol Cell Proteomics. 14(10), 2833-2847 (2015)]. After each selection, Fab-phage ELISAs were performed to determine the relative affinity and selectivity of these binders for their antigen targets (FIG. S2). Multiple antibodies were generated against each receptor, with a total of 69 antibodies isolated against six NK cell receptors (Table S1).

### Functional Screen Identifies Activating Antibodies

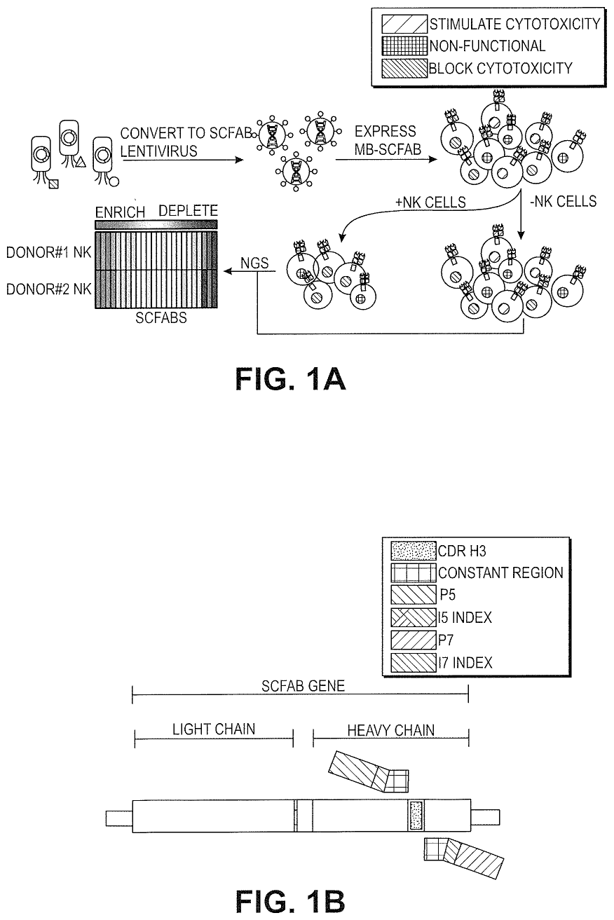

To evaluate the properties that are needed to generate effective NK cell engagers, we developed a pooled functional screen to assess the abilities of the selected antibody clones to induce NK cytotoxicity. The 69 antibodies that were generated, along with an anti-GFP control, were pooled and converted into single-chain Fabs (scFabs). These were displayed on a Jurkat cell line (FIG. 1A) to generate a small mammalian display library. The scFabs were robustly expressed at the cell surface, as determined by staining for human-specific Fab (FIG. S3). This scFab mammalian display library was incubated for either 4 or 24 hours in the presence or absence of either resting or IL-2-stimulated purified peripheral blood NK cells. We hypothesized that Jurkat cells displaying antibody clones that could stimulate NK cytotoxicity would be depleted from the library when in the presence of NK cells. This depletion could be quantified through NGS of the most unique CDR of the antibodies, CDR H3 (FIG. 1B).

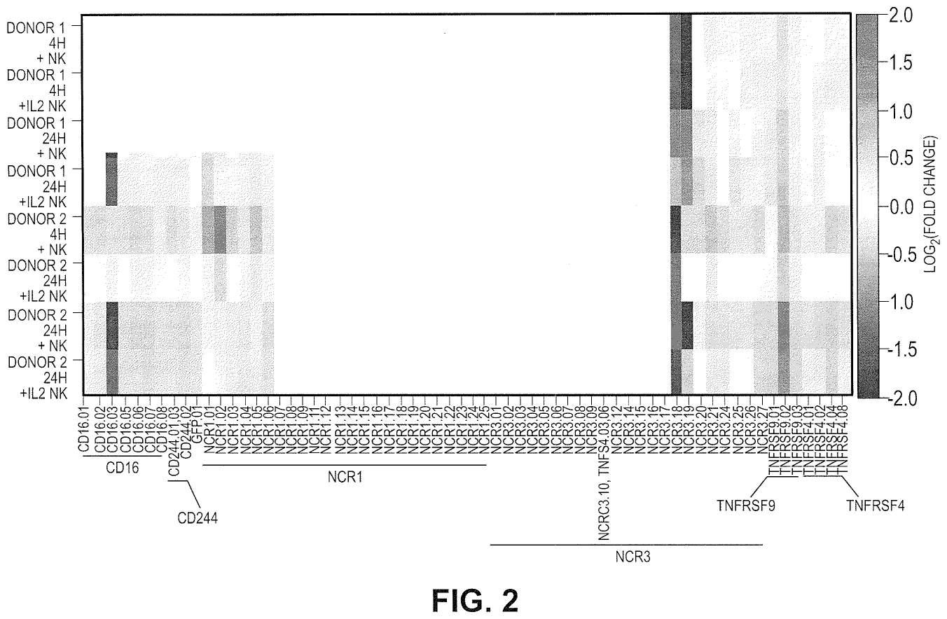

Because NK cells are highly heterogeneous and can vary greatly between individuals [Horowitz, A. et al., Sci. Transl. Med. 5(208), 208ra145 (2013); Strauss-Albee, D. M. et al., Sci. Transl. Med. 7(297), 297ra115 (2015)], we performed separate experiments using NK cells isolated from two different blood donors. Pairwise comparisons of the normalized NGS signals for the biological replicates showed good reproducibility; particularly at the 24-hour time point (FIG. S4). Although the results of the functional screen were not completely reproducible, particularly for the 4-hour time point by donor 2, pairwise comparisons of the normalized NGS signals for the biological replicates generally showed good reproducibility; particularly at the 24-hour time point (FIG. S4). Surprisingly, although all of the antibodies were generated to target NK cell surface proteins, only 4 antibodies, CD16.03, NCR1.11, NCR3.18, and NCR3.19 were depleted in the presence of NK cells (FIG. 2). Interestingly, only antibodies that targeted known activating receptors appeared to induce NK cell-mediated cytotoxicity. Many tumors have been known to overexpress stress-induced ligands that can stimulate NK cell activity [Raulet, D. H & Guerra, N., Nat. Rev. Immunol 9(8), 568-580 (2009); Marcus, A. et al., Adv. Immunol. 122, 91-128 (2014)]. Like many of these tumors, Jurkat cells have been shown to express NK cell ligands [Bae, D. S., Hwang, Y. K. & Lee, J. K., Cell. Immunol. 276(1-2), 122-127 (2012); Giuliani, E., Desimio, M. G. & Doria, M., Sci. Rep. 9(1), 4373 (2009)]. These ligands could potentially synergize with costimulatory receptor signaling to promote NK cell-mediated cytotoxicity. However, antibodies targeting costimulatory receptors or other cell surface proteins on NK cells were not depleted. This implies that even if tumors overexpress NK cell ligands, that the recruitment of NK cells through costimulatory receptors or other NK cell surface antigens may not be sufficient to drive tumor cell lysis. The stimulation of NK cell activating receptors may be needed for the development of effective NK cell-based therapeutics

### Validation of Antibody Hits Identified in Screen

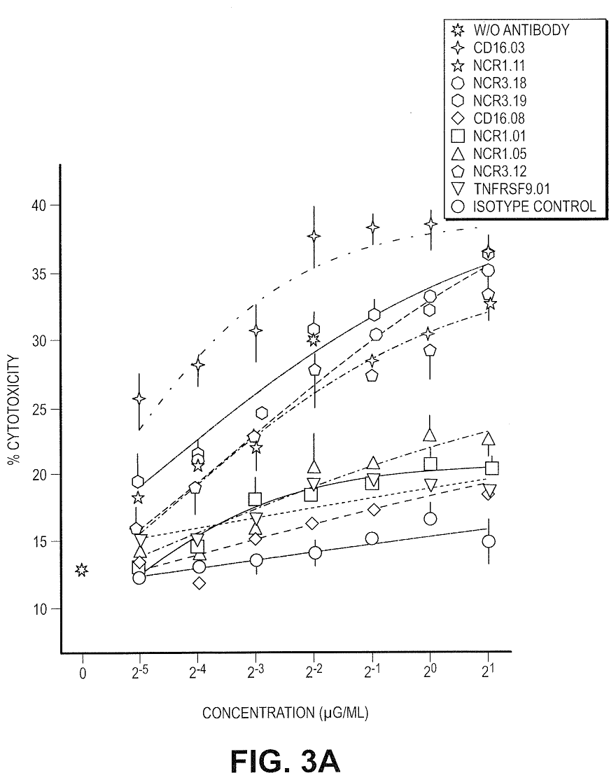

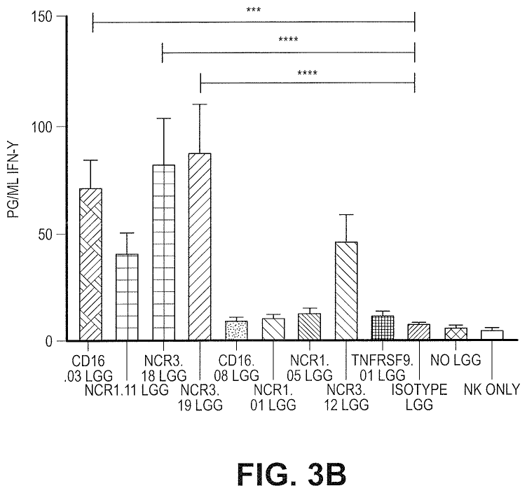

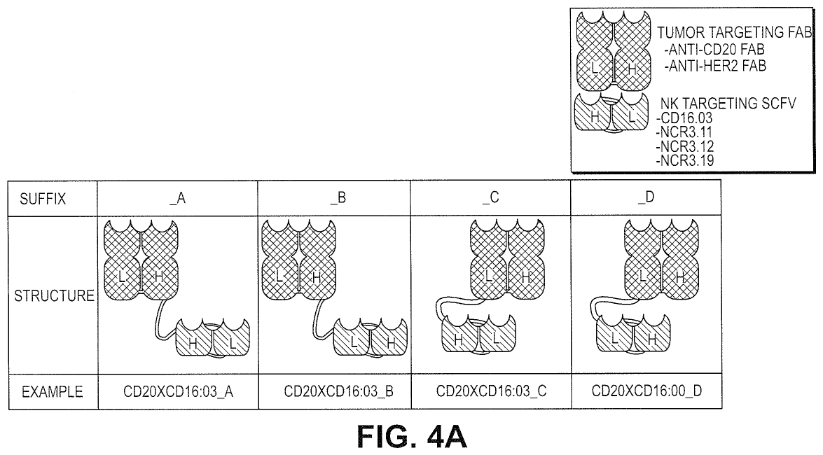

To validate the observations made by the functional screen, we chose to characterize the activity of nine antibody clones, four that were identified as activating and five that were identified as non-functional. We generated the IgG versions of these antibodies and tested their abilities to stimulate NK cell cytotoxicity in an antibody-redirected lysis assay (FIG. 3A). In this assay, FcγR⁺ THP-1 cells would bind to the Fc portion of the IgGs, and the Fab arms would bind the effector NK cells. If the Fab arms were able to stimulate NK cell-mediated cytotoxicity, then the THP-1 target cells would be lysed. Gratifyingly, all four antibodies that were identified as activating, CD16.03, NCR1.11, NCR3.18, and NCR3.19, were able to induce NK cytotoxicity. Moreover, four of the five antibodies that were identified as non-functional, CD16.08, NCR1.01, NCR1.05, and TNFRSF9.01, did not appear to stimulate NK cytotoxicity. However, one of the antibodies that was identified as non-functional, NCR3.12, appeared to stimulate NK cytotoxicity.

We also sought to determine if other NK cell effector functions, like cytokine secretion, could be stimulated with our antibodies. To determine if our antibodies were also able to induce cytokine secretion, we measured the amount of IFN-γ produced by NK cells that were co-incubated with FcγR⁺ P815 cells and IgG. Only the activating antibodies, CD16.03, NCR1.11, NCR3.18, and NCR3.19, and the putative non-functional antibody, NCR3.12, were able to significantly increase the amount of IFN-γ secreted (FIG. 3B). Although NCR3.12 was able to stimulate NK cell activity, it does not stimulate as much cytotoxicity or IFN-γ secretion as the other activating antibodies.

Activating Antibodies have High Affinity for their Receptor Targets

Although many of the antibodies target the same cell surface receptors, not every antibody was able to stimulate NK cell activity. To better understand the differences between activating and non-functional antibodies, we determined the specificity and affinity of the antibodies. To investigate the specificity of both activating and non-functional antibodies for their receptor targets, we developed a tetracycline-inducible cell line for each protein target—CD16, NCR1, NCR3, CD244, TNFRSF9, and TNFSF4. The ECDs of these proteins were fused to a generic transmembrane domain and were expressed upon tetracycline addition. Both activating and non-functional antibody clones bound exclusively to cells that overexpressed their respective receptor targets. No off-target binding was observed (FIG. S5), demonstrating that both activating and non-functional antibodies were highly selective for the receptor targets that they had been selected against.

We also determined if antibody affinity played a role in the difference between activating and non-functional antibodies. To evaluate the affinity of the selected antibodies for NK cells, we titrated the nine Fab clones on peripheral blood NK cells. For antibodies that bound to the activating receptors, CD16, NCR1, and NCR3, activating antibodies were found to bind more tightly to NK cells than non-functional antibodies (FIG. S6). Additionally, NCR3.12, the putative non-functional antibody that was able to stimulate NK cell activity had a much lower affinity than the activating antibodies, NCR3.18 and NCR3.19. This suggests that high affinity antibodies to activating receptors were better able to induce NK cytotoxicity, and that antibodies that were depleted in the functional screen were high affinity NK cell binders. Importantly, the non-functional antibody that bound to the costimulatory receptor, TNFRSF9, bound with high affinity yet displayed little to no NK cell activity. This further supported our functional screen results, which suggested that activating receptors must be targeted to induce NK cytotoxicity.

### Generation of Bispecific Antibodies Towards CD20+ B Cell Lymphoma Cells and HER2+ Breast Cancer Cells

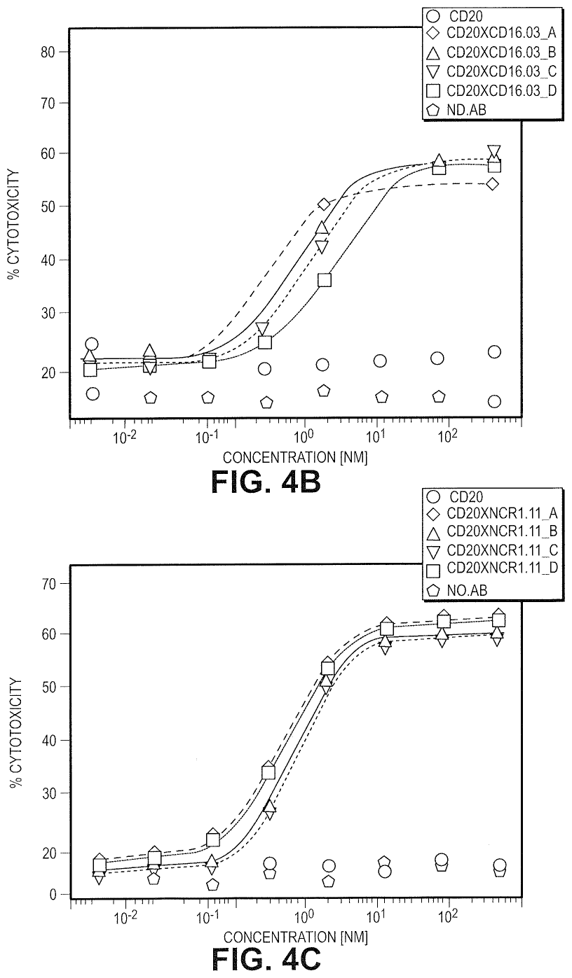

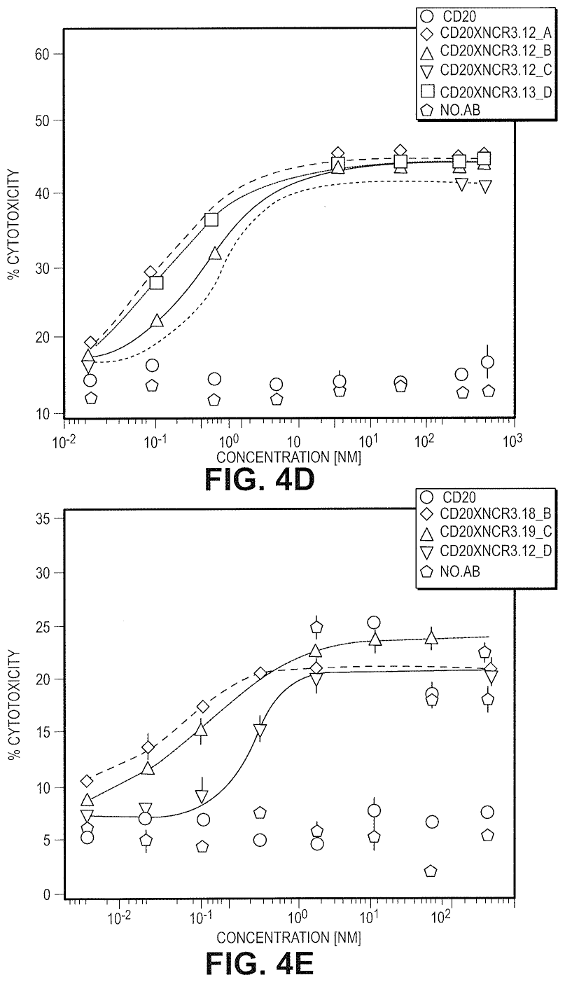

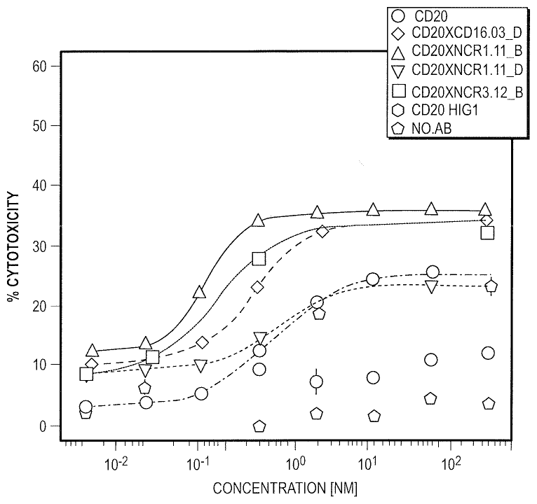

To demonstrate that these antibodies may be used to further the development of NK therapeutics, we generated CD20 targeting bispecific antibodies. CD16.03, NCR1.11, NCR3.12, and NCR3.19 were converted into single-chain variable fragments (scFvs) and associated with the anti-CD20 Rituximab Fab with a flexible linker. Additionally, to test if scFv domain ordering or Fab arm linkage has an effect on binding or stimulating cytotoxicity, we generated constructs with different domain orders, whether VH-VL (HL) or VL-VH (LH) and attached the scFv to either the heavy or light chain of the CD20 Fab (FIG. 4A). We then evaluated their ability to redirect NK cell-mediated cytotoxicity towards CD20+ Daudi B cell lymphoma cells. All of the bispecific constructs generated were able to bind to their respective antigens in a dose-dependent manner (FIGS. S7A-D), and promote the lysis of Daudi cells (FIG. 4B-E). Moreover, CD20×NCR3.12_B was as effective as the anti-CD20 human TgG1 mAb, and CD20×CD16.03_D, CD20×NCR1.11_B, and CD20×NCR1.11_D were found to be even more effective than the anti-CD20 human IgG1 mAb (FIG. 4F). This suggests that designing high affinity bispecific antibodies that target other activating receptors, like NCR1 or NCR3 may be as effective as, if not more effective than, antibodies inducing ADCC.

While all constructs were able to stimulate NK cytotoxicity, some subtle, but consistent differences were observed. Whereas almost all of the NCR1.11-based bispecific antibodies appeared to all be of somewhat equal efficacy, certain CD16.03-, NCR3.12-, and NCR3.19-based bispecific antibodies were more effective than others. Of the CD16.03-based bispecific antibodies, the LH domain ordering was more potent than their HL counterparts. Additionally, linkage of the CD16.03 scFv to the anti-CD20 light chain was effective than linkage to the heavy chain. In comparison, the CD20×NCR3.12_B bispecific antibody stimulated NK cytotoxicity better than any of the other NCR3.12-based bispecific antibody. Furthermore, of the NCR3.19-based bispecific antibodies, the bispecifics with the LH based domain order appeared to outperform the bispecific with a HL based domain order. Overall, LH ordering induced NK cell-mediated cytotoxicity more robustly than the HL ordering. However, differences in efficacy due to the linkage of the scFv to either the light chain or the heavy chain of the tumor targeting arm may be dependent on the NK cell targeting scFv.

To demonstrate the versatility of these constructs, we also generated HER2-targeting bispecific antibodies from NCR1.11. The NCR1.11 antibody was converted into an scFv and associated with the anti-HER2 Trastuzumab Fab. Again, constructs with different domain orders and attachment to either chain of the Fab were generated and their ability to lyse HER2+SK-BR3 breast cancer cells was evaluated. Once more, all of the constructs were able to redirect NK cell-mediated cytotoxicity towards SK-BR3 cells (FIG. S8), demonstrating that these antibodies can be reformatted to target different tumor cell types.

### Bispecific Antibodies Promote Cytotoxicity in Rituximab-Refractory B Cell Lymphoma Cells

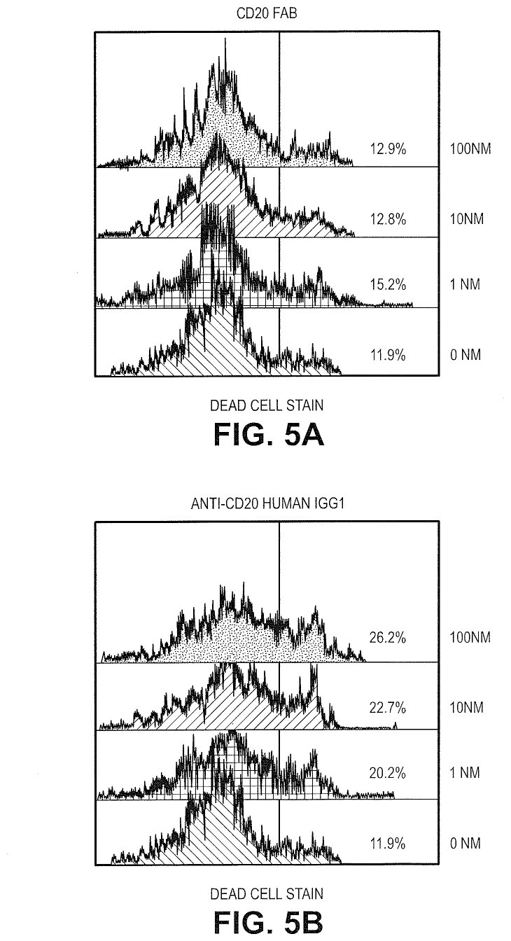

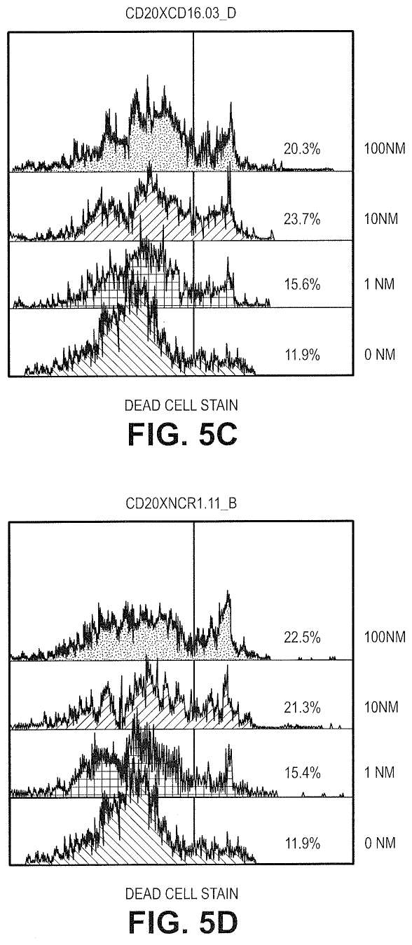

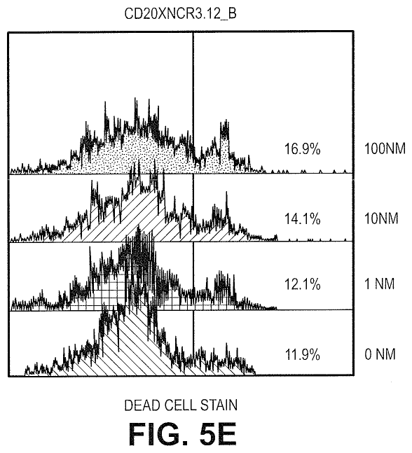

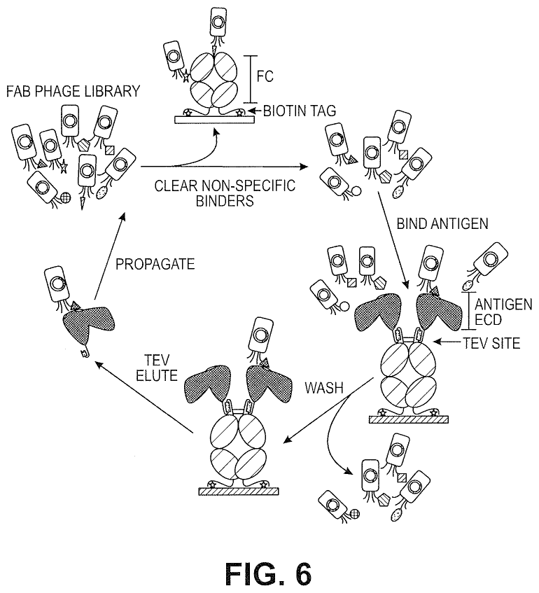

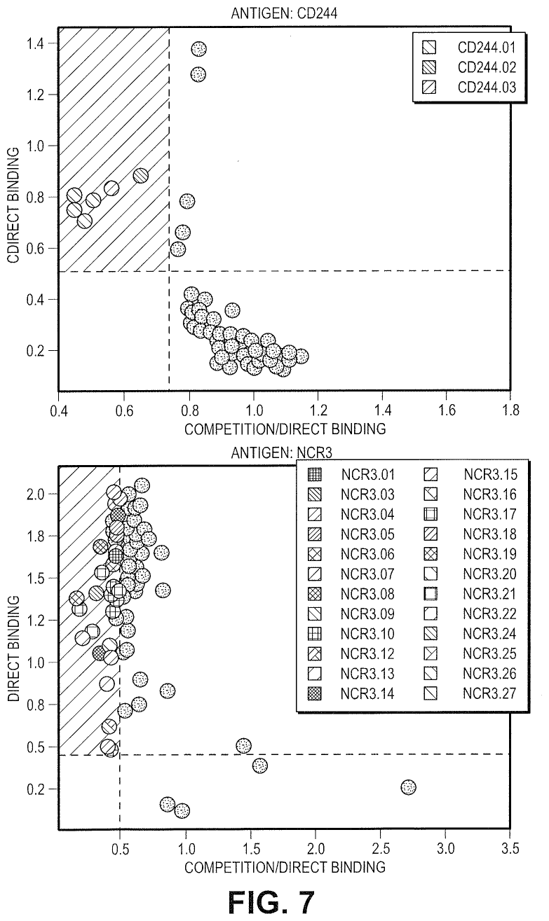

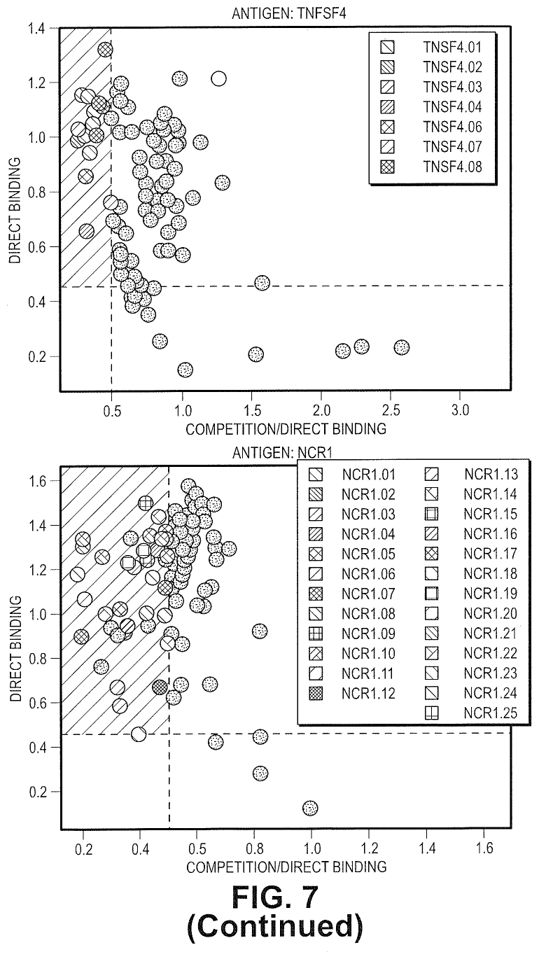

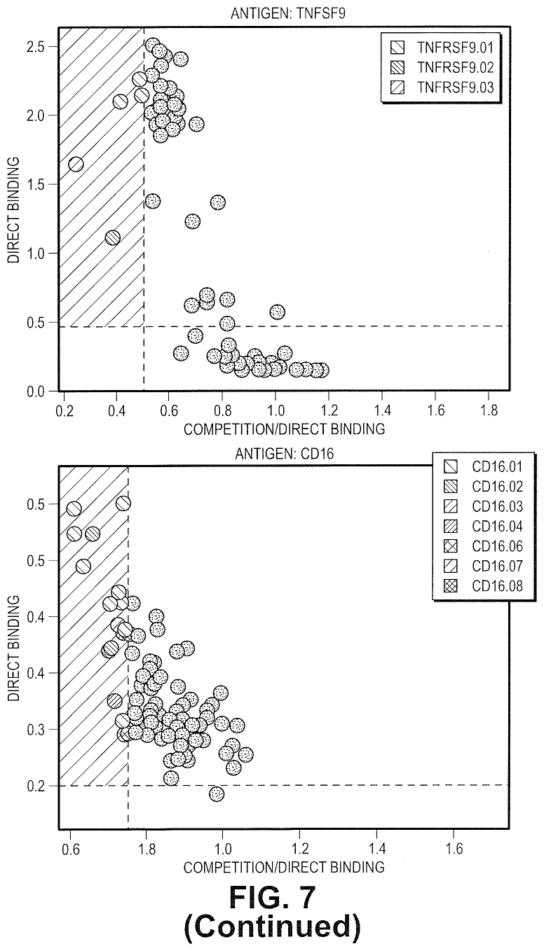

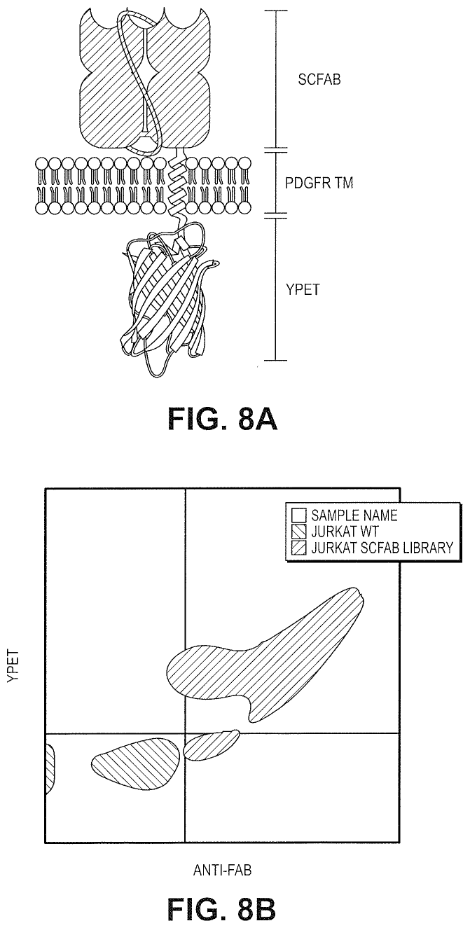

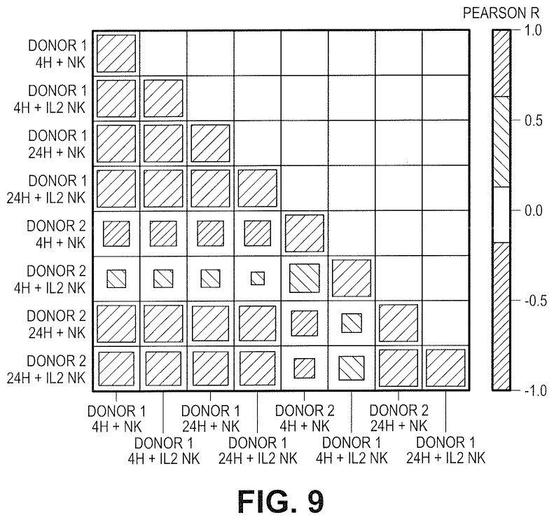

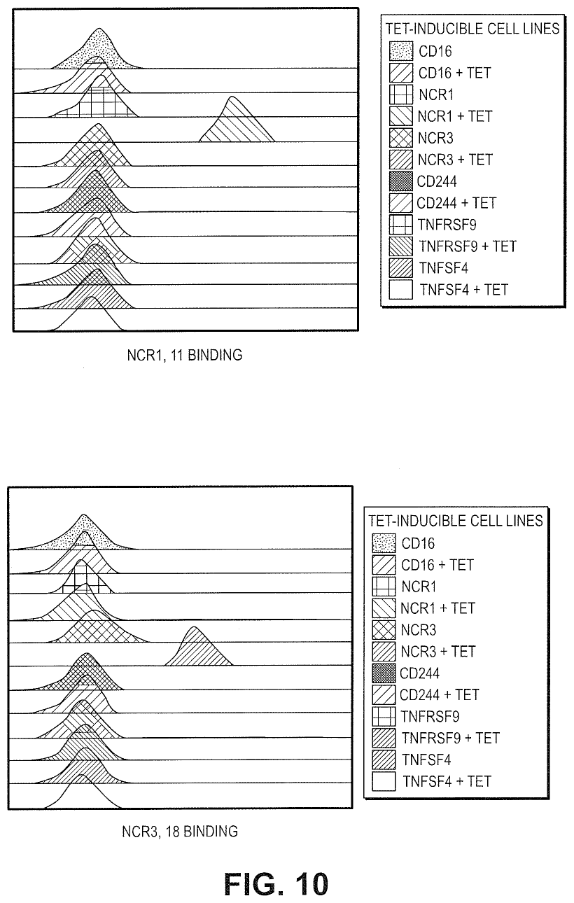

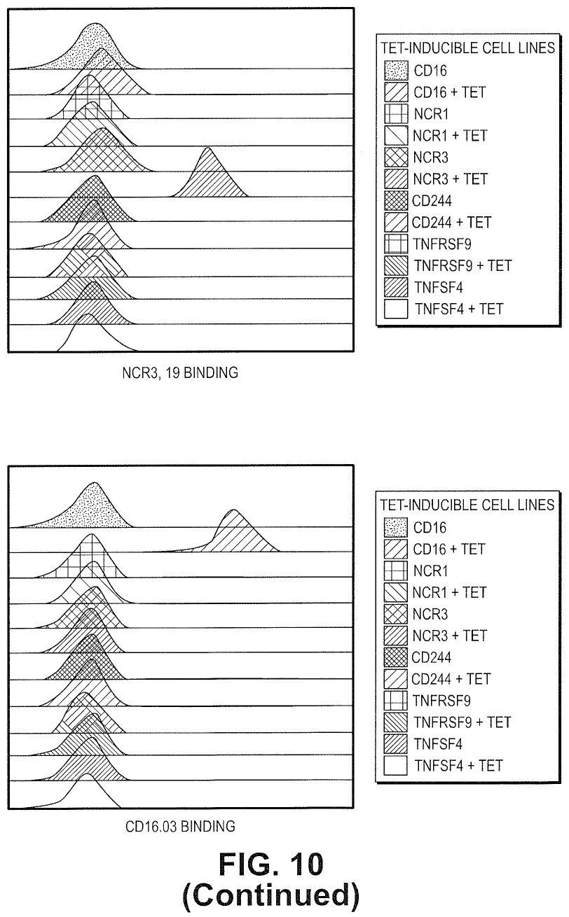

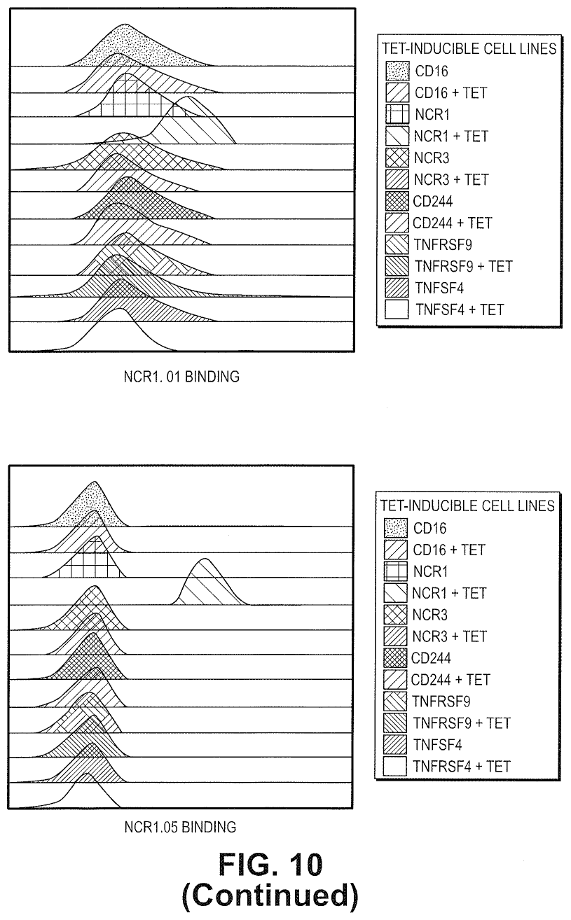

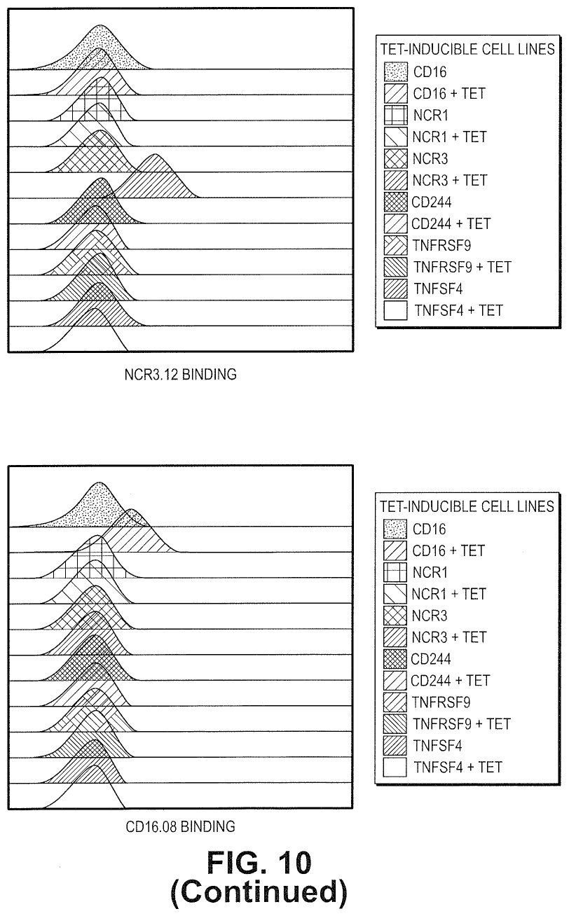

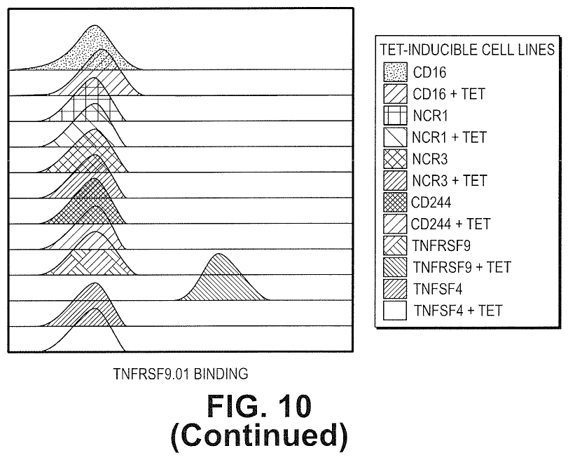

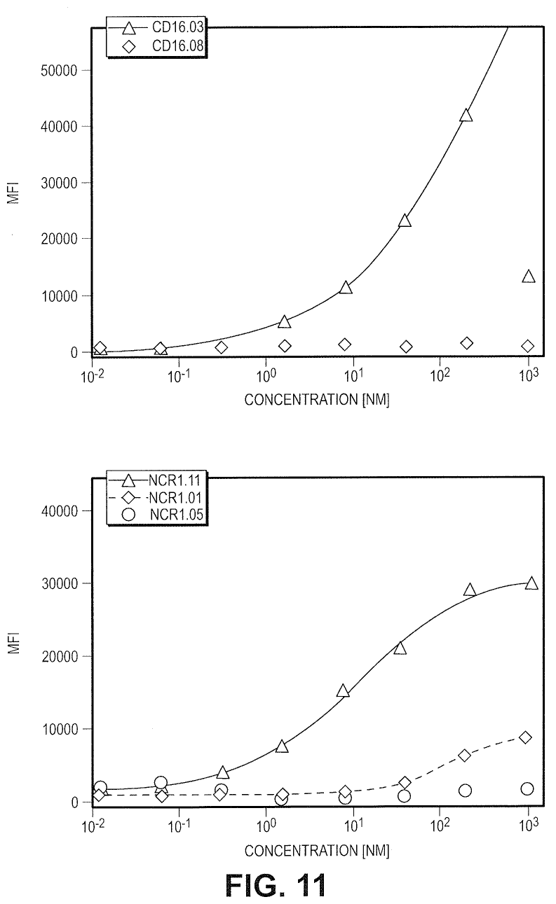

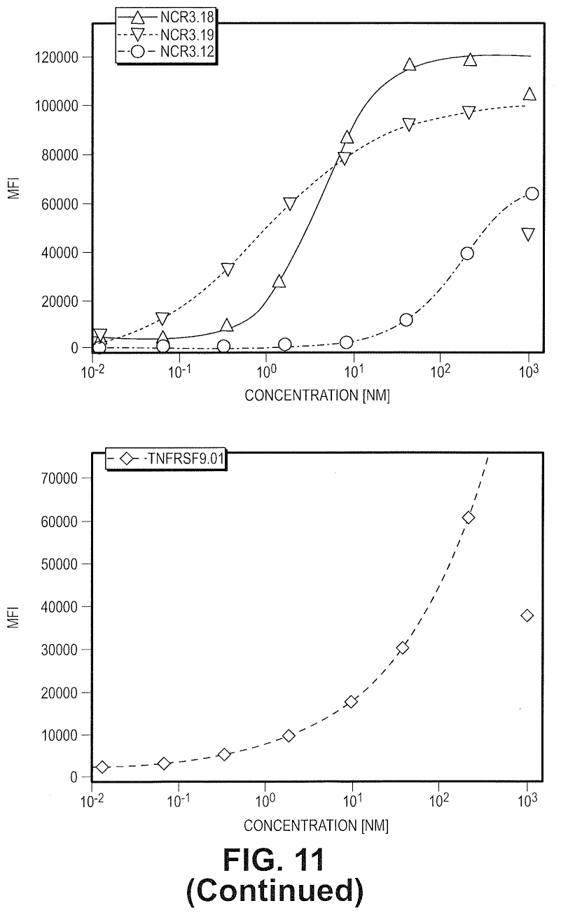

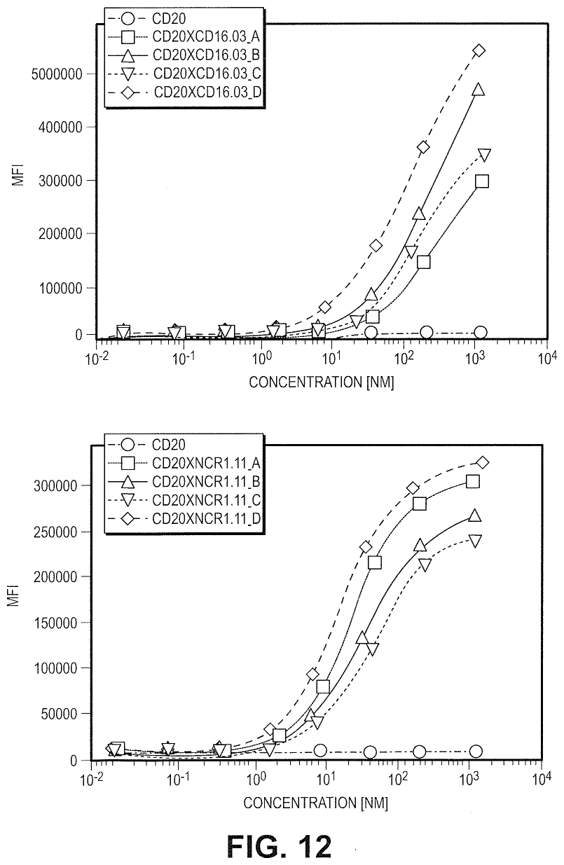

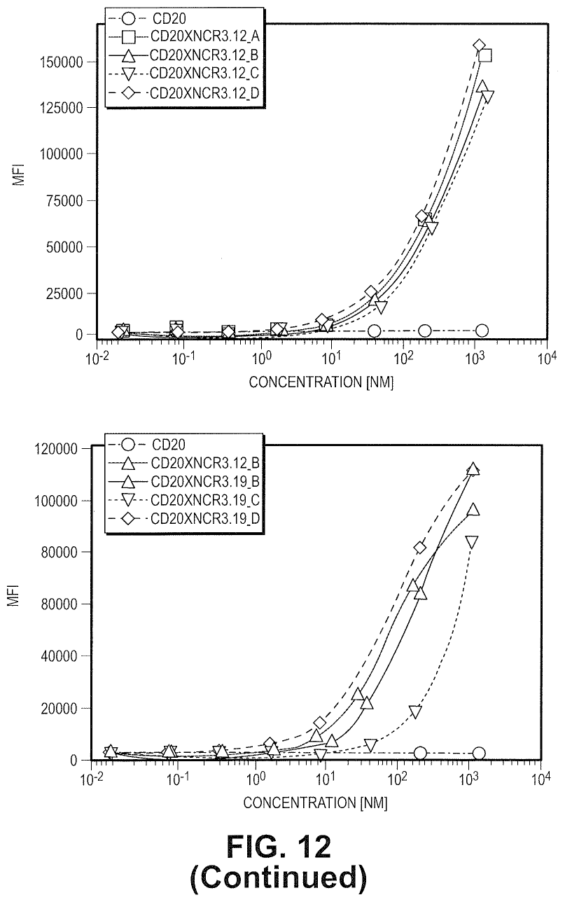

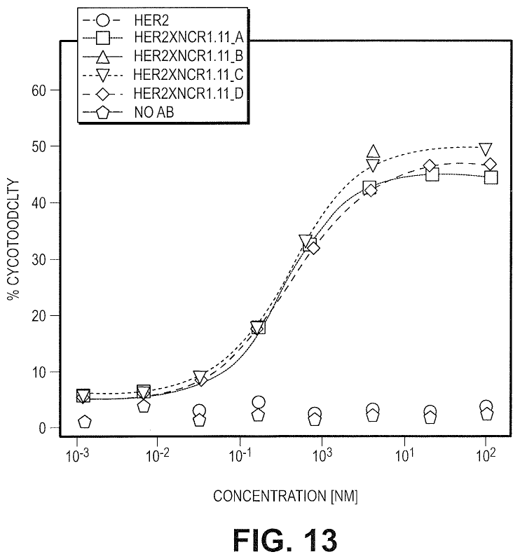

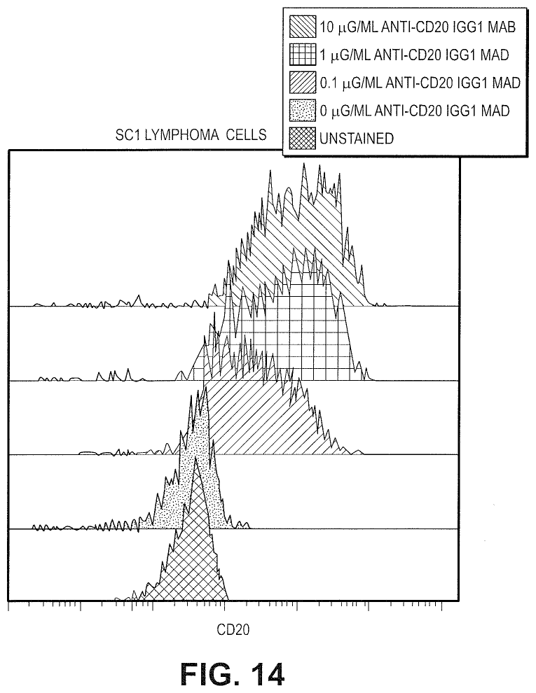

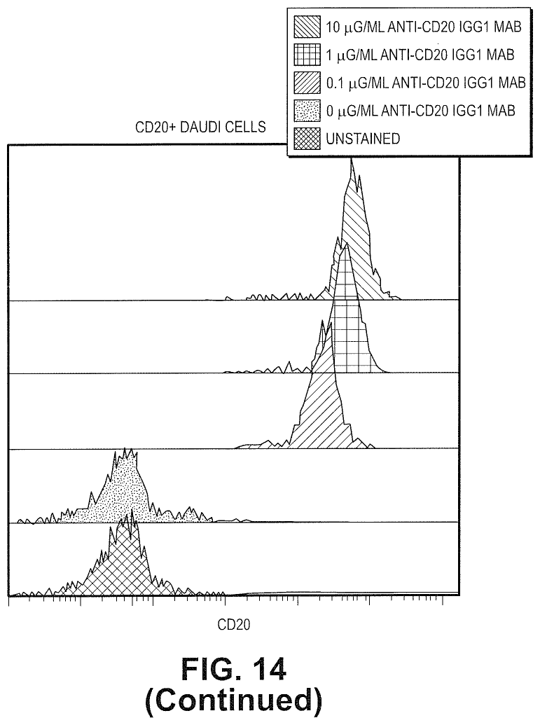

To determine if the bispecific antibodies generated would be able to redirect NK cell-mediated cytotoxicity towards primary B cell lymphomas, we tested the efficacy of three of the most potent bispecific antibodies, CD20×CD16.03_D, CD20×NCR1.11_B, and CD20×NCR3.12_B, against the SC1 lymphoma line. The SC1 cell line was derived from a patient with a highly refractory, CD79-mutated diffuse large B cell lymphoma, originating in skin and metastasizing to the brain and cerebrospinal fluid. The tumor was refractory to a combination rituximab plus cyclophosphamide, vincristine, adriamycin and prednisone, as well as to high-dose methotrexate plus rituximab. It was also refractory to combination etoposide plus cytarabine and to irradiation. All of the bispecific antibodies tested were able to redirect NK cell-mediated cytotoxicity towards SC1 lymphoma cells (FIG. 5). While the bispecific antibodies generated were more potent than the anti-CD20 human IgG1 mAb against the CD20+ Daudi cell line, the anti-CD20 human IgG1 mAb was slightly more effective against the SC1 lymphoma cells than the bispecific antibodies. The different cytotoxicity's observed between Daudi and SC1 lymphoma cells could be due to the different binding affinities of the bispecific antibodies and the anti-CD20 human IgG1 mAb towards the two different lymphoma cell lines. Indeed, CD20 expression levels are lower and more variable in the SC1 lymphoma cells than in the CD20+ Daudi cell line (FIG. S9A-B). In contrast to the bispecific antibodies that only have a single CD20-binding arm, the bivalent nature of the anti-CD20 human IgG1 mAb may allow the IgG to better bind to SC1 lymphoma cells.

### Discussion

NK cells have the unique ability to recognize and kill unhealthy cells, and are known to play a key role in cancer immunosurveillance. As such, they have become an attractive target for developing new cancer immunotherapies. In this study, we describe an approach to identify functional antibodies that can recruit and stimulate NK cell activity. From the hits identified from our mammalian display screen, we demonstrated the potential of generating various NK cell-targeting therapeutics by constructing bispecific antibodies to redirect NK cell-mediated cytotoxicity towards CD20+ lymphoma cells, as well as HER2+ breast cancer cells.

To facilitate the advancement of NK cell targeting therapeutics, we developed a functional mammalian display screen to rapidly assess the ability of a curated set of 69 antibodies to stimulate NK cytotoxicity. Others have previously used phage display [Reusch, U. et al., MAbs. 6(3), 728-739 (2014)] and hybridoma technology [Gauthier, L. et al., Cell. 177(7), 1701-1713 (2019)] to identify NK cell binders. Using mammalian display, we are able to assess these unique functional effects and rapidly identify clones for further investigation. Indeed, other groups have also used mammalian display to successfully identify individual antibody or peptide clones that induce unique phenotypes [Han, K. H. et al., Proc Natl Acad Sci USA. 115(3), E372-E381 (2018); Blanchard, J. W. et al., Nat Biotechnol. 35(10), 960-968 (2017)] or stimulate specific functional effects [Stepanov, A. V. et al., Sci Adv. 4(11), eaau4580 (2018)]. Inspired by such work, we created a functional screen to assess the unique cytotoxic effects of NK cells. Moreover, as our desired phenotype was amenable to the large sequencing capabilities of NGS, we were able to quantify the functional effects of all of our clones in parallel.

We developed multiple antibodies to target different NK cell surface proteins, including known activating receptors—CD16, NCR1, and NCR3, costimulatory receptors—TNFRSF9 and CD244, and an NK cell receptor with no known regulatory role—TNFSF4. Surprisingly, only four out of 69 antibodies were depleted from the mammalian display library by the introduction of NK cells, demonstrating the effectiveness of our screening method. Interestingly, all of these antibodies targeted known NK activating receptors, like CD16, NCR1, and NCR3. However, it should be noted that the majority of our antibodies in the functional screen target NCR1 and NCR3. This could potentially bias our functional screen towards antibodies that stimulate NCR1 or NCR3.

Upon further analysis, we determined that high affinity antibodies targeting activating receptors were able to stimulate NK cytotoxicity and IFN-γ secretion. This is consistent with previous findings that demonstrated that higher affinity CD16 polymorphisms were better able to mediate ADCC [Koene, H. R. et al., Blood. 90(3), 1109-1114 (1997); Wu, J. et al., J. Clin. Invest. 100(5), 1059-1070 (1997)] and were associated with a higher response rate to rituximab, trastuzumab, and cetuximab treatment [Weng, W. K. & Levy, R., J Clin Oncol. 21(21), 3940-3947 (2003). Musolino, A. et al., J Clin Oncol. 26(11), 1789-1796 (2008), Rodriguez, J. et al., Eur. J. Cancer. 48(12), 1774-1780 (2012)]. Although only a select number of antibodies were chosen for additional testing, the correlation that we found between antibody affinity and NK cell activity agrees with previous reports describing binders towards CD16 and NCR1. Others have previously shown that antibodies that bind to epitopes outside of the Fc-binding site of CD16 can stimulate ADCC, and that higher affinity CD16 binders are more potent than their lower affinity counterparts [Gleason, M. K et al., Mol Cancer Ther 11(12), 2674-2684 (2012); Ellwanger, K. et al., MAbs. 11(5), 899-918 (2019)] Additionally, NCR1 binding antibodies are able to stimulate NK cell-mediated cytotoxicity, regardless of which domain on NCR1 is targeted [Gauthier, L. et al., Cell. 177(7), 1701-1713 (2019)]. This suggests that high affinity antibodies are needed to stimulate NK cell activity.

Although developing high affinity antibodies towards NK cell receptors appears to be needed to stimulate NK cell activity, we have found that high affinity antibodies targeting other NK cell receptors, outside of known activating NK cell receptors, were not able to stimulate NK cell-mediated cytotoxicity. It is not entirely surprising that targeting costimulatory receptors did not result in NK cell activation as others have previously shown that NK cell activation typically requires co-engagement of different activating and costimulatory NK cell receptors [Bryceson, Y. T. et al., Blood. 107(1), 159-166 (2006); Bryceson, Y. T., Ljunggren, H. G. & Long, E. O., Blood. 114(13), 2657-2666 (2009)].

To demonstrate the utility of the antibodies identified by the functional screen, we converted four of the activating antibodies, CD16.03, NCR1.11, NCR3.12, and NCR3.19, into NK targeting bispecific antibodies. All of the CD20 targeting bispecific and the Her2 targeting bispecific antibodies generated were able to redirect NK cytotoxicity towards CD20+ Daudi B cell lymphoma cells and Her2+ SK-BR3 breast cancer cells, respectively. This suggests that the antibodies identified by the screen may be used to further develop different NK cell targeting therapeutics. Indeed, high affinity antibodies targeting CD16 [Gleason, M. K. et al., Mol Cancer Ther. 11(12), 2674-2684 (2012); Ellwanger, K. et al., MAbs. 11(5), 899-918 (2019)] and NCR1 [Gauthier, L. et al., Cell. 177(7), 1701-1713 (2019)] have previously been developed to create bispecific- and trispecific-NK cell engagers and redirect NK cell cytotoxicity, and appeared to have good efficacy in vitro and in vivo. In this study, several of the bispecific antibodies, including an NCR3 targeting bispecific antibody, were at least as potent as the anti-CD20 human IgG1 mAb, suggesting that developing antibodies against NCR3 may also be an effective way to recruit and stimulate NK activity.

In addition to promoting robust lysis of the well-established CD20+ Daudi B cell lymphoma cell line, our bispecific antibodies were also able to redirect NK cell cytotoxicity towards the highly refractory SC1 B cell lymphoma line. However, our bispecific antibodies were not any more efficacious than the anti-CD20 human IgG1 mAb in promoting the lysis of SC1 B cell lymphoma cells. This may be due to the avidity effect that the anti-CD20 human IgG1 mAb has towards CD20+ cells. Although our bispecific antibodies were not any more efficacious than the anti-CD20 human IgG1 mAb in this case, additional engineering to improve the affinity of the tumor-targeting moiety can further promote the cytotoxic potential of the bispecific antibodies developed. More importantly, the antibodies identified via our functional screen appear to be amenable towards the development of additional NK cell targeting engagers.

Given the growing interest in developing antibodies to target other immune cell types to the tumor microenvironment, we believe that this method is useful in identifying novel targets and antibodies that can redirect the cytotoxic or phagocytic functions of other immune cell types. The size of the mammalian display library can be increased to probe a larger set of immune cell receptors. Additionally, the same mammalian display library may be used to screen the functions of multiple immune cell types, so as to determine if certain subsets of antibodies may be used to cross-react with different cell types. Moreover, since all of these antibodies are based on the same scaffold, the desired antibody can be easily cloned and converted into different multi-specific formats. We believe that this work provides important insights into the design of NK cell-targeting antibodies and illustrates a novel method useful for identifying new immunotherapeutic antibodies.

### SI Materials and Methods

### Cells

HEK293T cells were cultured in DMEM supplemented with 100% FBS and 100 IU/mL penicillin and 100 μg/mL streptomycin. Jurkat and Raji cells were cultured in RPMI-1640 containing 2 mM Lglutamine containing 10% FBS and 100 IU/mL penicillin and 100 μg/mL streptomycin. NK92MI cells were cultured in α-MEM without ribonucleosides and deoxyribonucleosides, but containing 2 mM L-glutamine, and supplemented with 0.2 mM inositol, 0.1 mM 2-mercaptoethanol, 0.02 mM folic acid, 12.5% horse serum, 12.5% FBS, and 100 IU/mL penicillin and 100 μg/mL streptomycin. NKL cells that were stably transduced with NCR1 were maintained in RPMI-1640 containing 2 mM L-glutamine containing 10% FBS and 100 IU/mL penicillin and 100 μg/mL streptomycin, supplemented with 200 U/m. IL-2 (National Cancer Institute BRB Preclinical Repository). SK-BR3 cells were cultured in McCoy's 5a supplemented with 10% FBS and 100 IU/mL penicillin and 100 μg/mL streptomycin.

PBMCs were isolated by Ficoll-Paque and maintained in RPMI-1640 containing 2 mM L-glutamine containing 10% FBS and 100 IU/mL penicillin and 100 μg/mL streptomycin. Primary human NK cells were isolated from peripheral blood of de-identified, healthy donors (Blood Centers of the Pacific or Vitalant) using RosetteSep (StemCell) followed by Ficoll-Paque. The cells were maintained in RPMI-1640 containing 2 mM L-glutamine containing 10% FBS and 100 TU/mL penicillin and 100 μg/mL streptomycin. Tetracycline inducible cell lines overexpressing NK cell surface protein ECDs were generated by co-transfecting pOG44 vector with a construct encoding each ECD fused to the transmembrane domain of platelet-derived growth factor with a HA tag in the pcDNA5/FRT mammalian expression vector.

### Phage Display Selections

TEV-cleavable Fc-fusion proteins were expressed and biotinylated in Expi293F cells using the standard expression protocol. Media were harvested after 4 days of expression and protein was purified by protein A affinity chromatography. Phage selections were performed according to previously established protocols1 with the Fab-phage Library E2. Non-specific binders were depleted from the library by incubating the phage pool with Fc-domain immobilized on streptavidin beads. Fab phage were selected using biotinylated Fc-fusions that were captured on streptavidin-coated magnetic beads, and were released through TEV elution. Each selection consists of four rounds. With each round, decreasing amounts of Fc-fusions (1 μM, 100 nM, 10 nM, and 10 nM) were used. ELISAs were performed for 96 individual Fab-phage clones from the third or fourth rounds of selection to evaluate for affinity and selectivity. The best clones were pooled and converted to scFabs and subcloned to a lentiviral expression vector for further characterization with our functional screen.

### Fab Phage ELISAs

ELISAs were performed as previously described1. In brief, Maxisorp plates were coated with 10 μg/mL of Neutravidin overnight at 4° C. Biotinylated target antigen (20 nM) was captured on the Neutravidin coated plates for 30 min, and then exposed to a 1:5 dilution of phage supernatants for 30 min. Bound phage were detected via a horseradish-peroxidase-conjugated anti-phage monoclonal antibody (GE Lifesciences).

### Production of Lentivirus in HEK293T Cells

Lentivirus was produced by the transfection of 2.2×106 HEK 293T cells in T-25 flask, using 3 μg of lentiviral expression vector from the pooled scFab NK cell binders, 0.33 μg of pMD2 G, and 2.7 μg of pCMV-dR8.91, and 15 μL, of FuGENE HD transfection reagent (Promega) After 48 hr, cell supernatant was collected and cellular debris was removed by a 45-μm pore filter. Jurkat cells were transduced at an MOI<0.3.

Functional Screen of scFab Mammalian Display System

Freshly isolated NK cells were cultured in the presence or absence of 400 U/mL IL-2 for 16 hours. The scFab mammalian display library was washed and was incubated for 4 hr or 24 hr with 10 μg/mL DNase 1 while in the presence or absence of resting or IL-2-stimulated NK cells Surviving cells were collected and genomic DNA was isolated and used as a PCR template for NGS. The H3 sequence was amplified from the genomic DNA with flanking primers using Q5 DNA polymerase (NEB). The mix was thermocycled for 20 cycles. The amplicon was gel purified and submitted to the CZBiohub for analysis on a NextSeq (Illumina) with a custom sequencing primer (as shown).

TGAGGACACTGCCGTCTATTATTGTGCTCGC.

The .fastq.gz files were processed using Galaxy (https.//usegalaxy.org/). Sequencing artifacts were removed, and adapter sequences were clipped with a custom sequence (as shown):

TACTGGGGTCAAGGAACCCTGGTCAAGATCGGAAGAGCACACGTCTGAAC
TCCAGTCAC.

A FASTQ masker was applied when the quality score fell below 30. And sequence counts were exported for further analysis. Raw NGS counts for each condition was normalized to counts per million (CPM), and depletion of specific antibody clones was reported as the log 2 (fold change) between the library when in the presence or the absence on NK cells.

### IgG and Bispecific Antibody Expression

IgGs were expressed as previously described (Martinko, A. J. et al. Targeting RAS-driven human cancer cells with antibodies to upregulated and essential cell-surface proteins. Elife. 7, e31098 (2018)). In brief, Expi293 cells were transiently cotransfected with two pFUSE vectors containing the heavy and light chains of interest at a 1:1 ratio. For IgGs, the pFUSE vectors contained a Fab heavy chain was fused to a mouse IgG1 Fc or a Fab light chain. For the bispecific antibodies, the pFUSE vectors contained a Fab heavy chain or a Fab light chain that were fused to the scFv of interest. The ExpiFectamine 293 transfection kit was used for transfections as per manufacturer's instructions. Supernatants were harvested after 5-7 days of expression, and protein was purified by Protein A or Protein L affinity chromatography. Proteins were assessed by SDS-PAGE for purity and quality.

### Calcein Release Cell Cytotoxicity Assay

Calcein release cell cytotoxicity assays were performed as previously described (Neri, S., Mariani, E., Meneghetti, A., Cattini, L. & Facchini, A. Calcein-acetyoxymethyl Cytotoxicity Assay: Standardization of a Method Allowing Additional Analyses on Recovered Effector Cells and Supernatants. Clin Diagn Lab Immunol. 8(6), 1131-1135 (2001)). Target cells were washed and resuspended in to a final concentration of 1-5×106/mL and labeled in 15 μM calcein-AM for 30 min at 37° C. Cells were washed twice and coincubated with effector cells (purified NK cells, PBMCs, NK92MI, or NCR1+ NKL cells) at the indicated effector to target ratio in the presence of varying antibody concentrations in triplicate. Maximum lysis was induced with 1% Triton X-100. After 2 hours, supernatants were collected and calcein release was measured on an Infinite 200 Pro plate reader (Ex: 485±9 nm; Em: 530±20 nm). Specific lysis was calculated as 100×(experimental target cell release−target cell spontaneous release)/(maximum release−target cell spontaneous release).

### Fab Expression

Fabs were expressed as previously described (Elledge, S. K. et al. Systematic identification of engineered methionines and oxaziridines for efficient, stable, and site-specific antibody bioconjugation. Proc Natd Acad Sci USA. 117(11), 5733-5740 (2020)). In brief, C43 (DE3) Pro+E. coli were transformed with expression plasmids and were grown in TB autoinduction media at 37° C. for 6 hours. Incubation temperature was then reduced to 30° C. and were grown for an additional 16-18 hr. Cells were harvested by centrifugation and Fabs were purified by Protein A affinity chromatography. Fab purity and integrity was assessed by SDS-PAGE.

### On Cell Titration for Flow Cytometry

Titrations were performed on primary human NK cells or tetracycline induced overexpression cell lines as indicated. When tetracycline induced overexpression cell lines were used, cells were dosed with 10 μg/mL tetracycline for 2 days prior to staining. The starting concentration of each Fab or bispecific antibody was 1 μM, and serial 1:5 dilutions were performed. Antibodies were incubated with cells for 1 hr at 4° C., washed 2 times in 3% BSA in PBS pH 7.4, and stained for 30 min with an Alexafluor-647 conjugated goat anti-human IgG F(ab′)2 fragment (Jackson ImmunoResearch Laboratories) at a 1:50 dilution. After an additional 3 washes, cells were fixed and fluorescence was quantified using a CytoFLEX (Beckman Coulter). All flow cytometry data were analyzed using FlowJo software.

### Flow Cytometry-Based Cell Cytotoxicity Assay

Flow cytometry-based cell cytotoxicity assays were performed as previously described (Kandarian, F., Sunga, G. M., Arango-Saenz, D. & Rossetti, M. A Flow Cytometry-Based Cytotoxicity Assay for the Assessment of Human NK Cell Activity. J Vis Exp. 9(126), 56191 (2017)). In brief, target cells were stained with 1 μM CFSE for 20 min at 37° C. Cells were washed and coincubated with resting NK cells at the indicated effector to target ratio in the presence of varying antibody concentrations in duplicate. Maximum lysis was induced with 0.1% Tween-20. After 2 hours, dead cells were stained with 5 nM Sytox Red and fluorescence was quantified using a CytoFLEX (Beckman Coulter). All flow cytometry data were analyzed using FlowJo software.

### IFN-γ Secretion Assay

IFN-γ secretion was quantified with the ELISA max deluxe sets (BioLegend) according to the manufacturer's instructions. In brief, NK cells were incubated in the presence or absence of P815 target cells at an effector to target ratio of 1.1 with or without 1 μg/mL of each selected antibody for 24 hours. Supernatant was collected and assayed for IFN-□□content.

### Data Analysis

A one-way ANOVA with Dunnett's post hoc test was used for comparison of IFN-γ secretion induced by selected antibodies. Data were analyzed using GraphPad Prism 6.0 software. Dose response curves for the IgGs were fit with a three-parameter logistic model. Dose response curves for the bispecific antibodies were fit with a four-parameter logistic model using a python script.

### Further Information:

After affinity maturation 17 unique Fab clones were tested. Of the 17 clones, 16 expressed well enough for additional testing. We tested for binding to NCR3 via ELISA and on cell. When tested by ELISA, we determined that 13 clones had a lower EC50 than the parental NCR3.18 clone. Via on cell binding, we found that 8 clones had a lower EC50 than the parental NCR3.18 clone. To test for developability, we characterized the elution profile of the antibodies with SEC. Of the 17 clones, 11 clones maintained their SEC profile. Overall, we believe we have 5 high affinity clones with good elution profiles that can be used in bispecific or trispecific constructs.

| | | EC50 | EC50 | |
|---|---|---|---|---|
| | | (nM), | (nM), | Good |
| Fab name | HC3 | ELISA | on cell | SEC |
| NCR3.18.1 | RASSRFRSYYDSFYYAGM | 0.78 | 2.14 | + |
| NCR3.18.3 | RIGSIYRSYYDSFYYAGM | 0.68 | 1.61 | + |
| NCR3.18.4 | RISSHYMSYYDSFYYAGM | 2.29 | 1.44 | + |
| NCR3.18.6 | RISSSYMSYYDSFYYAGM | 1.38 | 4.67 | + |
| NCR3.18.7 | RISSYYISYYDSFYYAGM | 1.23 | 3.19 | + |
| NCR3.18.8 | RISSYYVSYYDSFYYAGM | 0.95 | 2.74 | + |
| NCR3.18.9 | RKSSSYWSYYDSFYYAGM | 0.51 | 2.24 | + |
| NCR3.18.10 | RKSSYYMSYYDSFYYAGM | 1.11 | 4.09 | + |
| NCR3.18.11 | RLGSRYRSYYDSFYYAGM | 0.77 | 1.47 | + |
| NCR3.18.12 | RRASYYKTYYDSFYYAGM | 0.53 | 2.13 | + |
| NCR3.18.13 | RRSSYYMTYYDSFYYAGM | 0.98 | 2.55 | + |
| NCR3.18.14 | RTGSYYMTYYDSFYYAGM | 0.57 | 3.11 | − |
| NCR3.18.15 | RTSSHYISYYDSFYYAGM | 2.98 | 6.76 | − |
| NCR3.18.16 | RTSSYYISYYDSFYYAGM | Low | Low | − |
| | | expres- | expres- | |
| | | sion | sion | |
| NCR3.18.17 | RVGSYYMSYYDSFYYAGM | 0.58 | 2.11 | − |
| NCR3.18.18 | RVSSNYMSYYDSFYYAGM | 2.2 | 3.39 | − |
| NCR3.18.19 | RVSSPYMSYYDSFYYAGL | 0.38 | 1.41 | − |
| NCR3.18 | RISSYYMSYYDSFYYAGM | 1.63 | 2.27 | + |

TABLE S1

| Name | Antigen | LC3 | HC1 | HC2 | HC3 |
|---|---|---|---|---|---|
| CD16.01 | CD16 | YRGGRLI | VYSYYI | YISPSSGSTY | GYYSAWRYSMGL |
| CD16.02 | CD16 | AMYWELL | VSSSYI | SIYSYYGYTS | SYSYYSTWYMHRYFYAAF |
| CD16.03 | CD16 | SSAELI | FSSYSI | SIYSSSGSTS | WSYDQYYDQHGYYFYYWGF |
| CD16.05 | CD16 | WPGSLV | FYYYSI | SIYPSSGSTS | WSYHGM |
| CD18.06 | CD16 | SSSSLI | FSSSSI | SISSSSGSTS | FPYRWSYYWYSYYKWYAM |
| CD16.07 | CD16 | YGQRNPI | FYSSYI | SISPYSGYTY | SWYYFSAM |
| CD16.08 | CD16 | SSHHLI | FSSSSI | SIYPSYGYTS | TYSYYAM |
| CD244.01 | CD244 | SVGVPV | LSYSSI | SIYSYYGYTS | YYEYYGYGYAAM |
| CD244.02 | CD244 | SYNYSPL | ISYYSI | SIYPYYGYTS | NYYLYYGM |
| CD244.03 | CD244 | SVGVPV | ISYYSI | SIYSYYGYTS | YYEYYGYGYAAM |
| NCR1.01 | NCR1 | SYYYDLV | FSSSSI | SIYSYYGYTS | YKFKNAYWWSYYNTQGVGM |
| NCR1.02 | NCR1 | SMYHQLI | VSYSSI | SIYSYYGSTS | YSFEPSLYSYYWDSRSAF |
| NCR1.03 | NCR1 | WWSSPI | VSSYSI | SIYSSYGSTY | KMGWYYYWYDGL |
| NCR1.04 | NCR1 | SYYYYLV | FSYSYI | SISSSSGYTS | HYYRDFYQWTGGM |
| NCR1.05 | NCR1 | SWAWPV | FSYSSI | SIYSSYGSTY | FLGGAM |
| NCR1.06 | NCR1 | SYFPPI | LYSSSI | SIYPYYSSTS | SDGYWYWYGWYWGM |
| NCR1.07 | NCR1 | STYEPI | VYYSSI | SISSYSSYTS | EVHWYRYSYWYYYLTQAM |
| NCR1.08 | NCR1 | NWYYPLI | VYYSSI | SISPYSGYTS | MYNYQMYWLFGGGYGM |
| NCR1.08 | NCR1 | SSGWYPL | FSSYSI | SIYSYYGYTS | SESYWPYGWYGF |
| NCR1.10 | NCR1 | SSSSLI | LSSYSI | SISSYYSSTS | SWPYSSQQWYYYYYWYAM |
| NCR1.11 | NCR1 | SSSSLI | VYYSY! | SISSYYGSTY | SRYLQDYWSSWWVSWYGL |
| NCR1.12 | NCR1 | SSSSLI | FSSSSI | SISSSSGSTS | SSMWSFPGGWQHYSFGM |
| NCR1.18 | NCR1 | SSYSLV | FYSSSI | SIYPYYGYTS | PWQPAYYGYAYGM |
| NCR1.14 | NCR1 | SSSSLI | VYSSSI | SISPYYGYTS | TGQYYYSYVYGL |
| NCR1.15 | NCR1 | GYENPL | VYSSYI | SIYSSYGYTS | YAFSLPSMWWYSYSGM |
| NCR1.16 | NCR1 | SWYYPI | FSSSSI | SISSSSGSTS | SYVGRYSYYYYPGMGI |
| NCR1.17 | NCR1 | SAHYPV | FSYSYI | SIYSSSSSTY | EGLGWTWGAWLYWYGGI |
| NCR1.18 | NCR1 | SGWSSPI | VYSSSI | SISPSYGYTS | DILFFSYWYSAL |
| NCR1.19 | NCR1 | SSYHPL | FSYSYI | SISSSSGSTS | WNTMYYYWYRPWGSWAM |
| NCR1.20 | NCR1 | SWHSSSLI | VSYSSI | SIYSYYGYTS | EYQQSYWYKWAL |
| NCR1.21 | NCR1 | GWYFTLI | VYYSSI | SISSSYGSTS | YPRSYFWGMVSRTQGL |
| NCR1.22 | NCR1 | VRFGYPV | VYSSSI | SISSSSGSTS | SDLQQYWWWPSYYGYGGF |
| NCR1.23 | NCR1 | SYVSPI | LYSSSI | YISSSYGYTY | DMYKPGWYYYWGSGDYGF |
| NCR1.24 | NCR1 | SYAWPV | VYSYSI | SISSYSGYTY | NSWLAGTMSAYRYQGL |
| NCR1.25 | NCR1 | YYFKPI | VYSSSI | SISSYSSSTY | NHYYAQSYGIYQYFSGF |
| NCR3.01 | NCR3 | SSYEYPV | VYYSSI | SIYPSSGSTY | WYIKWDTWYYSDAM |
| NCR3.02 | NCR3 | SYGSLI | VSSYSI | SIYPYYGSTS | WYQYYYQYSLGF |
| NCR3.03 | NCR3 | SYGFLI | VYSYSI | SIYPYSGYTS | WYNYHHWWSRAF |
| NCR3.04 | NCR3 | SGRQLV | VSSYSI | SIYSYYGSTY | WYQYYPSQGGMAM |
| NCR3.05 | NCR3 | SSWYYPF | LYSYSI | SIYPYYGSTS | WYQYWDWSYSRGAM |
| NCR3.08 | NCR3 | FSLSLL | VYYSSI | SISPSYGYTS | SYSYGFAM |
| NCR3.07 | NCR3 | SFGFYYPF | VYYSSI | SIYPYSGYTS | DYTSNSYGDYYYYGGYGF |
| NCR3.08 | NCR3 | SGYYWPI | FSSSSI | SISSSSGSTS | WLMWFSYAHGAYHMPYGL |
| NCR3.09 | NCR3 | SSKYLV | VYSYSI | YIYPYSGSTS | WFTYHWPGSIAF |
| NGR3.10 | NCR3 | SSYEYPV | VYYSSI | SISPYYGYTS | WYQYSDSIAM |
| NCR3.12 | NCR3 | SSYWPF | ISSSSI | YISSSSGYTS | YSYFYGGYFYWTSWGAF |
| NCR3.13 | NCR3 | YYGYLI | VYSYSI | SISPYYGYTS | WYQYSDSIAM |
| NCR3.14 | NCR3 | SSSSLI | FYSYSI | SISSSSGSTS | YFYDSPYYTYFSNYPSAL |
| NCR3.15 | NCR3 | SSSSLI | VYSSSI | SISSSSGSTS | YVGPSYHSVWYYYSWYYAI |
| NCR3.16 | NCR3 | SWESLV | VSYSSI | SISPYYGYTS | YHDRYTYYFGYYGWSYGM |
| NCR3.17 | NCR3 | HWRVSLI | VSSYSI | SIYSYSGSTS | WYMYYYNSDYFGL |
| NCR3.18 | NCR3 | SSSSLI | VSSSSI | SISSSSGSTS | RISSYYMSYYDSFYYAGM |
| NCR3.19 | NCR3 | QWYPLI | VYSYSI | SIYSYYGSTS | WYQYYYIGTAAM |
| NCR3.20 | NCR3 | SSSSLI | LSYSSI | SISSSSGSTS | RYKGIVWWSYWSNWYYMGL |
| NCR3.21 | NCR3 | KGNRLI | ISYSSI | SIYSYYSSTS | YSGGYFLFYTYDYYVYGF |
| NCR3.22 | NCR3 | SSSSLI | VYSYSI | SISSSSGSTS | WYGYYYYGEFAF |
| NCR3.23 | NCR3 | SSYGLF | VSSYSI | SISPYYGYTS | WYQYSDSIAM |
| NCR3.24 | NCR3 | TSSLPI | VYSSSI | SISPYSGYTY | WYMYYYKIAM |
| NCR3.25 | NCR3 | SSSSLI | VSSYSI | SISSYYGSTS | QWEYSSYHYSTWYYAWAM |
| NCR3.26 | NCR3 | SSSSLI | ISYSSI | SISSSSGSTS | RYDSYYWYYYSSYWYYGM |
| NCR3.27 | NCR3 | LYSRLV | VSYSSI | SIYPYSGYTS | PYDYYGWYWGAF |
| TNFRSF9.01 | TNFRSF9 | GSWYSGLI | LYSSSI | SIYCSYGSTY | NWYWYAL |
| TNFRSF9.02 | INFRSF9 | SYYYDPV | IYSSSI | SIYSYSGSTY | EFVQWHYGYFYYDDWYAF |
| TNFRSF9.03 | TNFRSF9 | SSLGYLI | VSSYSI | SISSYYGYTS | EWWEGWAL |
| TNFSF4.01 | TNFSF4 | TSSSYLI | VYSYSI | SIYPYSGSTS | WYQYSRPRDWGL |
| TNFSF4.02 | TNFSF4 | GYSSSSLI | IYSYSI | SIYPYYSYTS | WYMYAEADAM |
| TNFSF4.04 | TNFSF4 | SWVSGPF | LSSYSI | SIYSYSGYTS | WYHHFYHMAM |
| TNFSF4.06 | TNFSF4 | YYGYLI | VYSYSI | SISPLLWLYF | WYQYSDSIAM |
| TNFSF4.08 | TNFSF4 | SYSDSSLF | VYSYSI | SIYPSYGSTY | WYGYSYYSHEAM |
| GFP.01 | GFF | SWGLI | ISYYSI | SIYPYYSSTS | AGWVASSGM |

### Sequences

All antibodies are based on the Trastuzumab scaffold. Only CDR L3, H1, H2, and H3 are varied. The numbering is based on the IMGT scheme. CDRs are underlined in the variable chain sequences.

anti-NCR3 Fab #72
Light chain:
SEQ ID NO: 1:
DIQMTQSPSSLSASVGDRVTITCRASQSVSSAVAWYQQKPGKAPKLLIYSASSLYSGV
PSRFSGSRSGTDFTLTISSLQPEDFATYYCQQSSYWPFTFGQGTKVEIKRTVAAPSVFI
FPPSDSQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 2: LCDR1 RASQSVSSAV
SEQ ID NO: 3: LCDR2 SASSLYS
SEQ ID NO: 4: LCDR3 SSYWPF
Heavy chain
SEQ ID NO: 5:
EVQLVESGGGLVQPGGSLRLSCAASGFNISSSSIHWVRQAPGKGLEWVAYISSSSGY
TSYADSVKGRFTISADTSKNTAYLQMNSLRAEDTAVYYCARYSYFYGGYFYWTSWGA
FDYWGQGTLVTVSSASTKGPSVFPLAPSSKSTSGGTAALGOLVKDYFPEPVTVSWNS
GALTSGVHTFPAVLQSSGLYSLSSVVTVPSSSLGTQTYICNVNHKPSNTKVDKKVEPK
SCDKTHT
SEQ ID NO: 6: HCDR1 ISSSSI
SEQ ID NO: 7: HCDR2 YISSSSGYTS
SEQ ID NO: 8: HCDR3 YSYFYGGYFYWTSWGAF
anti-NCR3 Fab #78
Light chain:
SEQ ID NO: 9:
DIQMTQSPSSLSASVGDRVTITCRASQSVSSAVAWYQQKPGKAPKLLIYSASSLYSGV
PSRFSGSRSGTDFTLTISSLQPEDFATYYCQQSSSSLITFGQGTKVEIKRTVAAPSVFIF
PPSDSQLKSGTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 10: LCDR1 RASQSVSSAV
SEQ ID NO: 11: LCDR2 SASSLYS
SEQ ID NO: 12: LCDR3 SSSSLI
Heavy chain
SEQ ID NO: 13:
EVQLVESGGGLVQPGGSLRLSCAASGFNVSSSSIHWVRQAPGKGLEWVASISSSSGS
TSYADSVKGRFTISADTSKNTAYLQMNSLRAEDTAVYYCARRISSYYMSYYDSFYYAG
MDYWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKVEPK
SCDKTHT
SEQ ID NO: 14: HCDR1 VSSSSI
SEQ ID NO: 15: HCDR2 SISSSSGSTS
SEQ ID NO: 16: HCDR3 RISSYYMSYYDSFYYAGM
anti-NCR3 Fab #79
Light chain:
SEQ ID NO: 17:
DIQMTQSPSSLSASVGDRVTITCRASQSVSSAVAWYQQKPGKAPKLLIYSASSLYSGV
PSRFSGSRSGTDFTLTISSLQPEDFATYYCQQQWYPLITFGQGTKVEIKRTVAAPSVFI
FPPSDSQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 18: LCDR1 RASQSVSSAV
SEQ ID NO: 19: LCDR2 SASSLYS
SEQ ID NO: 20: LCDR3 QWYPLI
Heavy chain
SEQ ID NO: 21:
EVQLVESGGGLVQPGGSLRLSCAASGFNVYSYSIHWVRQAPGKGLEWVASIYSYYGS
TSYADSVKGRFTISADTSKNTAYLQMNSLRAEDTAVYYCARWYQYYYIGTAAMDYWG
QGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTH
T
SEQ ID NO: 22: HCDR1 VYSYSI
SEQ ID NO: 23: HCDR2 SIYSYYGSTS
SEQ ID NO: 24: HCDR3 WYQYYYIGTAAM
anti-NCR1 Fab #27
Light chain:
SEQ ID NO: 25:
DIQMTQSPSSLSASVGDRVTITCRASQSVSSAVAWYQQKPGKAPKLLIYSASSLYSGV
PSRFSGSRSGTDFTLTISSLQPEDFATYYCQQSSSSLITFGQGTKVEIKRTVAAPSVFIF
PPSDSQLKSGTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 26: LCDR1 RASQSVSSAV
SEQ ID NO: 27: LCDR2 SASSLYS
SEQ ID NO: 28: LCDR3 SSSSLI
Heavy chain
SEQ ID NO: 29:
EVQLVESGGGLVQPGGSLRLSCAASGFNVYYSYIHWVRQAPGKGLEWVASISSYYGS
TYYADSVKGRFTISADTSKNTAYLQMNSLRAEDTAVYYCARSRYLQDYWSSWWWVSW
YGLDYWGQGTLVTVSSASTKGPSVFPLAPSSKSTSGGTAALGOLVKDYFPEPVTVSW
NSGALTSGVHTFPAVLQSSGLYSLSSVVTVPSSSLGTQTYICNVNHKPSNTKVDKKVE
PKSCDKTHT
SEQ ID NO: 30: HCDR1 VYYSYI
SEQ ID NO: 31: HCDR2 SISSYYGSTY
SEQ ID NO: 32: HCDR3 SRYLQDYWSSWWVSWYGL
anti-CD16 Fab #3
Light chain:
SEQ ID NO: 33:
DIQMTQSPSSLSASVGDRVTITCRASQSVSSAVAWYQQKPGKAPKLLIYSASSLYSGV
PSRFSGSRSGTDFTLTISSLQPEDFATYYCQQSSAELITFGQGTKVEIKRTVAAPSVFIF
PPSDSQLKSGTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 34: LCDR1 RASQSVSSAV
SEQ ID NO: 35: LCDR2 SASSLYS
SEQ ID NO: 36: LCDR3 SSAELI
Heavy chain
SEQ ID NO: 37:
EVQLVESGGGLVQPGGSLRLSCAASGFNFSSYSIHWVRQAPGKGLEWVASIYSSSGS
TSYADSVKGRFTISADTSKNTAYLQMNSLRAEDTAVYYCARWSYDQYYDQHGYYFYY
WGFDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVE
PKSCDKTHT
SEQ ID NO: 38: HCDR1 FSSYSI
SEQ ID NO: 39: HCDR2 SIYSSSGSTS
SEQ ID NO: 40: HCDR3 WSYDQYYDQHGYYFYYWGF
Summary of anti-NCR3 Fab #78 CDR H3 sequences:
SEQ ID NO: 41: RX(G/A/S)SX(Y/F)X(S/T)YYDSFYYAG(M/L)
X can be any amino acid.
Additional specific alternate CDR H3 sequences of anti-NCR3 Fab #78:
SEQ ID NO: 42: RASSRFRSYYDSFYYAGM
SEQ ID NO: 43: RIGSIYRSYYDSFYYAGM
SEQ ID NO: 44: RISSHYMSYYDSFYYAGM
SEQ ID NO: 45: RISSSYMSYYDSFYYAGM
SEQ ID NO: 46: RISSYYISYYDSFYYAGM
SEQ ID NO: 47: RISSYYVSYYDSFYYAGM
SEQ ID NO: 48: RKSSSYWSYYDSFYYAGM
SEQ ID NO: 49: RKSSYYMSYYDSFYYAGM
SEQ ID NO: 50: RLGSRYRSYYDSFYYAGM
SEQ ID NO: 51: RRASYYKTYYDSFYYAGM
SEQ ID NO: 52: RRSSYYMTYYDSFYYAGM
SEQ ID NO: 53: RTGSYYMTYYDSFYYAGM
SEQ ID NO: 54: RTSSHYISYYDSFYYAGM
SEQ ID NO: 55: RVGSYYMSYYDSFYYAGM
SEQ ID NO: 56: RVSSNYMSYYDSFYYAGM
SEQ ID NO: 57: RVSSPYMSYYDSFYYAGL
In light chain, underlined sequences encode CDR L1, L2, L3
In heavy chain, underlined sequences encode CDR H1, H2, H3
anti-NCR3 Fab #72
Light chain:
SEQ ID NO: 58:
gatatccagatgacccagtccccgagctccctgtccgcctctgtgggcgatagggtcaccatcacctgccgtgccagtcag
tccgtgtccagcgctgtagcctggtatcaacagaaaccaggaaaagctccgaagcttctgatttactcggcatccagcctct
actctggagtcccttctcgcttctctggtagccgttccgggacggatttcactctgaccatcagcagtctgcagccggaagac
ttcgcaacttattactgtcagcaaTCTTCTTACTGGCCGTTCacgttcggacagggtaccaaggtggagatca
aacgaactgtggctgcaccatctgtcttcatcttcccgccatctgattcacagttgaaatctggaactgcctctgttgtgtgcctg
ctgaataacttctatcccagagaggccaaagtacagtggaaggtggataacgccctccaatcgggtaactcccaggaga
gtgtcacagagcaggacagcaaggacagcacctacagcctcagcagcaccctgacgctgagcaaagcagactacga
aaaacataaagtctacgcctgcgaagtcacccatcagggcctgagctcgcccgtcacaaagagcttcaacaggggag
agtgt
Heavy chain:
SEQ ID NO: 59:
gaggttcagctggtggagtctggcggtggcctggtgcagccagggggctcactccgtttgtcctgtgcagcttctggcttcaa
cATCTCTTCTTCTTCTATAcactgggtgcgtcaggccccgggtaagggcctggaatgggttgcaTATATTT
CTTCTTCTTCTGGCTATACTTCTtatgccgatagcgtcaagggccgtttcactataagcgcagacacatcca
aaaacacagcctacctacaaatgaacagcttaagagctgaggacactgccgtctattattGTGCTCGCTACTCT
TACTTCTACGGTGGTTACTTCTACTGGACTTCTTGGGGTGCTTTTGACTACTGgggtc
aaggaaccctggtcaccgtctcctcggcctccaccaagggtccatcggtcttccccctggcaccctcctccaagagcacct
ctgggggcacagcggccctgggctgcctggtcaaggactacttccccgaaccggtgacggtgtcgtggaactcaggcgc
cctgaccagcggcgtgcacaccttcccggctgtcctacagtcctcaggactctactccctcagcagcgtggtgaccgtgcc
ctccagcagcttgggcacccagacctacatctgcaacgtgaatcacaagcccagcaacaccaaggtcgacaagaaag
ttgagcccaaatcttgtgacaaaactcacaca
anti-NCR3 Fab #78
Light chain:
SEQ ID NO: 60:
gatatccagatgacccagtccccgagctccctgtccgcctctgtgggcgatagggtcaccatcacctgccgtgccagtcag
tccgtgtccagcgctgtagcctggtatcaacagaaaccaggaaaagctccgaagcttctgatttactcggcatccagcctct
actctggagtcccttctcgcttctctggtagccgttccgggacggatttcactctgaccatcagcagtctgcagccggaagac
ttcgcaacttattactgtcagcaatcttcttcttctctgatcacgttcggacagggtaccaaggtggagatcaaacgaactgtg
gctgcaccatctgtcttcatcttcccgccatctgattcacagttgaaatctggaactgcctctgttgtgtgcctgctgaataacttc
tatcccagagaggccaaagtacagtggaaggtggataacgccctccaatcgggtaactcccaggagagtgtcacagag
caggacagcaaggacagcacctacagcctcagcagcaccctgacgctgagcaaagcagactacgaaaaacataaa
gtctacgcctgcgaagtcacccatcagggcctgagctcgcccgtcacaaagagcttcaacaggggagagtgt
Heavy chain:
SEQ ID NO: 61:
gaggttcagctggtggagtctggcggtggcctggtgcagccagggggctcactccgtttgtcctgtgcagcttctggcttcaa
cGTCTCTTCTTCTTCTATAcactgggtgcgtcaggccccgggtaagggcctggaatgggttgcatctatttcttct
tcttctggctctacttcttatgccgatagcgtcaagggccgtttcactataagcgcagacacatccaaaaacacagcctacct
acaaatgaacagcttaagagctgaggacactgcggtctattattGTGCTCGCCGTATCTCTTCTTACTAC
ATGTCTTACTACGACTCTTTCTACTACGCTGGTATGGACTACTGgggtcaaggaaccctggt
caccgtctcctcggcctccaccaagggtccatcggtcttccccctggcaccctcctccaagagcacctctgggggcacag
cggccctgggctgcctggtcaaggactacttccccgaaccggtgacggtgtcgtggaactcaggcgccctgaccagcgg
cgtgcacaccttcccggctgtcctacagtcctcaggactctactccctcagcagcgtggtgaccgtgccctccagcagcttg
ggcacccagacctacatctgcaacgtgaatcacaagcccagcaacaccaaggtcgacaagaaagttgagcccaaatc
ttgtgacaaaactcacaca
anti-NCR3 Fab #79
Light chain:
SEQ ID NO: 62:
gatatccagatgacccagtccccgagctccctgtccgcctctgtgggcgatagggtcaccatcacctgccgtgccagtcag
tccgtgtccagcgctgtagcctggtatcaacagaaaccaggaaaagctccgaagcttctgatttactcggcatccagcctct
actctggagtcccttctcgcttctctggtagccgttccgggacggatttcactctgaccatcagcagtctgcagccggaagac
ttcgcaacttattactgtcagcaaCAGTGGTACCCGCTGATCacgttcggacagggtaccaaggtggagatca
aacgaactgtggctgcaccatctgtcttcatcttcccgccatctgattcacagttgaaatctggaactgcctctgttgtgtgcctg
ctgaataacttctatcccagagaggccaaagtacagtggaaggtggataacgccctccaatcgggtaactcccaggaga
gtgtcacagagcaggacagcaaggacagcacctacagcctcagcagcaccctgacgctgagcaaagcagactacga
aaaacataaagtctacgcctgcgaagtcacccatcagggcctgagctcgcccgtcacaaagagcttcaacaggggag
agtgt
Heavy chain:
SEQ ID NO: 63:
gaggttcagctggtggagtctggcggtggcctggtgcagccagggggctcactccgtttgtcctgtgcagcttctggcttcaa
cGTCTATTCTTATTCTATAcactgggtgcgtcaggccccgggtaagggcctggaatgggttgcaTCTATTT
ATTCTTATTATGGCTCTACTTCTtatgccgatagcgtcaagggccgtttcactataagcgcagacacatcca
aaaacacagcctacctacaaatgaacagcttaagagctgaggacactgccgtctattattGTGCTCGCTGGTAC
CAGTACTACTACATCGGTACTGCTGCTATGGACTACTGgggtcaaggaaccctggtcaccgtct
cctcggcctccaccaagggtccatcggtcttccccctggcaccctcctccaagagcacctctgggggcacagcggccctg
ggctgcctggtcaaggactacttccccgaaccggtgacggtgtcgtggaactcaggcgccctgaccagcggcgtgcaca
ccttcccggctgtcctacagtcctcaggactctactccctcagcagcgtggtgaccgtgccctccagcagcttgggcaccca
gacctacatctgcaacgtgaatcacaagcccagcaacaccaaggtcgacaagaaagttgagcccaaatcttgtgacaa
aactcacaca
anti-NCR1 Fab #27
Light chain:
SEQ ID NO: 64:
gatatccagatgacccagtccccgagctccctgtccgcctctgtgggcgatagggtcaccatcacctgccgtgccagtcag
tccgtgtccagcgctgtagcctggtatcaacagaaaccaggaaaagctccgaagcttctgatttactcggcatccagcctct
actctggagtcccttctcgcttctctggtagccgttccgggacggatttcactctgaccatcagcagtctgcagccggaagac
ttcgcaacttattactgtcagcaaTCTTCTTCTTCTCTGATCacgttcggacagggtaccaaggtggagatcaa
acgaactgtggctgcaccatctgtcttcatcttcccgccatctgattcacagttgaaatctggaactgcctctgttgtgtgcctgc
tgaataacttctatcccagagaggccaaagtacagtggaaggtggataacgccctccaatcgggtaactcccaggagag
tgtcacagagcaggacagcaaggacagcacctacagcctcagcagcaccctgacgctgagcaaagcagactacgaa
aaacataaagtctacgcctgcgaagtcacccatcagggcctgagctcgcccgtcacaaagagcttcaacaggggaga
gtgt
Heavy chain:
SEQ ID NO: 65:
gaggttcagctggtggagtctggcggtggcctggtgcagccagggggctcactccgtttgtcctgtgcagcttctggcttcaa
cGTCTATTATTCTTATATAcactgggtgcgtcaggccccgggtaagggcctggaatgggttgcaTCTATTT
CTTCTTATTATGGCTCTACTTATtatgccgatagcgtcaagggccgtttcactataagcgcagacacatcca
aaaacacagcctacctacaaatgaacagcttaagagctgaggacactgccgtctattattGTGCTCGCTCTCGT
TACCTGCAGGACTACTGGTCTTCTTGGTGGGTTTCTTGGTACGGTTTGGACTACTG
gggtcaaggaaccctggtcaccgtctcctcggcctccaccaagggtccatcggtcttccccctggcaccctcctccaagag
cacctctgggggcacagcggccctgggctgcctggtcaaggactacttccccgaaccggtgacggtgtcgtggaactca
ggcgccctgaccagcggcgtgcacaccttcccggctgtcctacagtcctcaggactctactccctcagcagcgtggtgac
cgtgccctccagcagcttgggcacccagacctacatctgcaacgtgaatcacaagcccagcaacaccaaggtcgacaa
gaaagttgagcccaaatcttgtgacaaaactcacaca
anti-CD16 Fab #3
Light chain:
SEQ ID NO: 66:
gatatccagatgacccagtccccgagctccctgtccgcctctgtgggcgatagggtcaccatcacctgccgtgccagtcag
tccgtgtccagcgctgtagcctggtatcaacagaaaccaggaaaagctccgaagcttctgatttactcggcatccagcctct
actctggagtcccttctcgcttctctggtagccgttccgggacggatttcactctgaccatcagcagtctgcagccggaagac
ttcgcaacttattactgtcagcaaTCTTCTGCTGAACTGATCacgttcggacagggtaccaaggtggagatca
aacgaactgtggctgcaccatctgtcttcatcttcccgccatctgattcacagttgaaatctggaactgcctctgttgtgtgcctg
ctgaataacttctatcccagagaggccaaagtacagtggaaggtggataacgccctccaatcgggtaactcccaggaga
gtgtcacagagcaggacagcaaggacagcacctacagcctcagcagcaccctgacgctgagcaaagcagactacga
aaaacataaagtctacgcctgcgaagtcacccatcagggcctgagctcgcccgtcacaaagagcttcaacaggggag
agtgt
Heavy chain:
SEQ ID NO: 67:
gaggttcagctggtggagtctggcggtggcctggtgcagccagggggctcactccgtttgtcctgtgcagcttctggcttcaa
cTTCTCTTCTTATTCTATAcactgggtgcgtcaggccccgggtaagggcctggaatgggttgcaTCTATTT
ATTCTTCTTCTGGCTCTACTTCTtatgccgatagcgtcaagggccgtttcactataagcgcagacacatcca
aaaacacagcctacctacaaatgaacagcttaagagctgaggacactgccgtctattattGTGCTCGCTGGTCT
TACGACCAGTACTACGACCAGCATGGTTACTACTTCTACTACTGGGGTTTTGACTA
CTGgggtcaaggaaccctggtcaccgtctcctcggcctccaccaagggtccatcggtcttccccctggcaccctcctcca
agagcacctctgggggcacagcggccctgggctgcctggtcaaggactacttccccgaaccggtgacggtgtcgtggaa
ctcaggcgccctgaccagcggcgtgcacaccttcccggctgtcctacagtcctcaggactctactccctcagcagcgtggt
gaccgtgccctccagcagcttgggcacccagacctacatctgcaacgtgaatcacaagcccagcaacaccaaggtcga
caagaaagttgagcccaaatcttgtgacaaaactcacaca

What is claimed is:

1. An antibody that specifically binds to human Natural Cytotoxicity Triggering Receptor 3 (NCR3), wherein the antibody comprises at least
(1) a light chain variable region comprising a light chain complementarity determining region (LCDR) 1 comprising SEQ ID NO:2, a LCDR2 comprising SEQ ID NO:3 and a LCDR3 comprising SEQ ID NO:4; and
a heavy chain variable region comprising a heavy chain complementarity determining region (HCDR) 1 comprising SEQ ID NO: 6, a HCDR2 comprising SEQ ID NO:7 and a HCDR3 comprising SEQ ID NO:8; or
(2) a light chain variable region comprising a light chain complementarity determining region (LCDR) 1 comprising SEQ ID NO:10, a LCDR2 comprising SEQ ID NO:11 and a LCDR3 comprising SEQ ID NO: 12; and
a heavy chain variable region comprising a heavy chain complementarity determining region (HCDR) 1 comprising SEQ ID NO: 14, a HCDR2 comprising SEQ ID NO:15 and a HCDR3 comprising SEQ ID NO:41; or
(3) a light chain variable region comprising a light chain complementarity determining region (LCDR) 1 comprising SEQ ID NO: 18, a LCDR2 comprising SEQ ID NO: 19 and a LCDR3 comprising SEQ ID NO:20; and
a heavy chain variable region comprising a heavy chain complementarity determining region (HCDR) 1 comprising SEQ ID NO: 22, a HCDR2 comprising SEQ ID NO:23 and a HCDR3 comprising SEQ ID NO:24.

2. The antibody of claim 1, comprising
a light chain variable region comprising a light chain complementarity determining region (LCDR) 1 comprising SEQ ID NO: 10, a LCDR2 comprising SEQ ID NO:11 and a LCDR3 comprising SEQ ID NO:12; and
a heavy chain variable region comprising a heavy chain complementarity determining region (HCDR) 1 comprising SEQ ID NO: 14, a HCDR2 comprising SEQ ID NO: 15 and a HCDR3 comprising SEQ ID NO:41.

3. The antibody of claim 2, wherein the HCDR3 comprises one of SEQ ID NO:16, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, or SEQ ID NO:57.

4. The antibody of claim 1, wherein,
the light chain variable region comprises SEQ ID NO:1; and
the light chain variable region comprises SEQ ID NO:5.

5. The antibody of claim 1, wherein,
the light chain variable region comprises SEQ ID NO:9; and
the light chain variable region comprises SEQ ID NO: 13.

6. The antibody of claim 1, wherein,
the light chain variable region comprises SEQ ID NO: 17; and
the light chain variable region comprises SEQ ID NO:21.

7. The antibody of any one of claims 1-6, wherein the antibody is a bi-specific antibody that binds NCR3 and a second target protein.

8. The antibody of claim 7, wherein the second target protein is expressed on cancer cells.

9. The antibody of claim 7, wherein the second target protein is CD20 or BCMA or HER2.

10. A polynucleotide encoding the antibody of any one of claims 1-9.

11. A cell that expresses the antibody of any one of claims 1-9.

12. The cell of claim 11, wherein the cell is a mammalian cell.

13. A method of stimulating natural killer (NK) cell-mediated cytotoxicity in a human in need thereof, the method comprising administering the antibody of any one of claims 8-9 to the human in an amount sufficient to stimulate NK cell-mediated cytotoxicity.

14. The method of claim 13, wherein the human has cancer and the NK cell-mediated cytotoxicity kills cancer cells.

15. The method of claim 14, wherein the cancer is multiple myeloma, leukemia, Hodgkin's lymphoma or non-Hodgkin's lymphoma.

16. An antibody that specifically binds to human Natural Cytotoxicity Triggering Receptor 1 (NCR1), wherein the antibody comprises at least
a light chain variable region comprising a light chain complementarity determining region (LCDR) 1 comprising SEQ ID NO: 26, a LCDR2 comprising SEQ ID NO:27 and a LCDR3 comprising SEQ ID NO:28; and
a heavy chain variable region comprising a heavy chain complementarity determining region (HCDR) 1 comprising SEQ ID NO: 30, a HCDR2 comprising SEQ ID NO:31 and a HCDR3 comprising SEQ ID NO:32.

17. The antibody of claim 16, wherein,
the light chain variable region comprises SEQ ID NO:25; and
the light chain variable region comprises SEQ ID NO:29.

18. The antibody of any one of claims 16-17, wherein the antibody is a bi-specific antibody that binds NCR1 and a second target protein.

19. The antibody of claim 18, wherein the second target protein is expressed on cancer cells.

20. The antibody of claim 18, wherein the second target protein is CD20 or BCMA or HER2.

21. A polynucleotide encoding the antibody of any one of claims 16-20.

22. A cell that expresses the antibody of any one of claims 16-20.

23. The cell of claim 11, wherein the cell is a mammalian cell.

24. A method of stimulating natural killer (NK) cell-mediated cytotoxicity in a human in need thereof, the method comprising administering the antibody of any one of claims 19-20 to the human in an amount sufficient to stimulate NK cell-mediated cytotoxicity.

25. The method of claim 24, wherein the human has cancer and the NK cell-mediated cytotoxicity kills cancer cells.

26. The method of claim 25, wherein the cancer is multiple myeloma, leukemia, Hodgkin's lymphoma or non-Hodgkin's lymphoma.

27. An antibody that specifically binds to human CD-16, wherein the antibody comprises at least
a light chain variable region comprising a light chain complementarity determining region (LCDR) 1 comprising SEQ ID NO: 34, a LCDR2 comprising SEQ ID NO:35 and a LCDR3 comprising SEQ ID NO:36; and
a heavy chain variable region comprising a heavy chain complementarity determining region (HCDR) 1 comprising SEQ ID NO: 38, a HCDR2 comprising SEQ ID NO:39 and a HCDR3 comprising SEQ ID NO:40.

28. The antibody of claim 27, wherein,
the light chain variable region comprises SEQ ID NO:25, and
the light chain variable region comprises SEQ ID NO:29.

29. The antibody of any one of claims 27-28, wherein the antibody is a bi-specific antibody that binds CD-16 and a second target protein.

30. The antibody of claim 29, wherein the second target protein is expressed on cancer cells.

31. The antibody of claim 29, wherein the second target protein is CD20 or BCMA or HER2.

32. A polynucleotide encoding the antibody of any one of claims 27-31.

33. A cell that expresses the antibody of any one of claims 27-31.

34. The cell of claim 11, wherein the cell is a mammalian cell.

35. A method of stimulating natural killer (NK) cell-mediated cytotoxicity in a human in need thereof, the method comprising administering the antibody of any one of claims 30-31 to the human in an amount sufficient to stimulate NK cell-mediated cytotoxicity.

36. The method of claim 35, wherein the human has cancer and the NK cell-mediated cytotoxicity kills cancer cells.

37. The method of claim 36, wherein the cancer is multiple myeloma, leukemia, Hodgkin's lymphoma or non-Hodgkin's lymphoma.

38. A method of identifying antibodies that activate natural killer (NK) cells, the method comprising,
providing a library of antibodies that bind to proteins on NK cells;
expressing the library of antibodies on the surface of mammalian cells;
incubating a population of the mammalian cells with NK cells under conditions in which the NK cells kill at least some mammalian cells based on the antibody expressed on the cells; and
following the incubating, quantifying the proportion of cells remaining;
comparing the proportion of cells remaining to a control population of mammalian cells, wherein a decrease in the proportion of cells expressing a particular antibody indicates the particular antibody activates NK cells.

39. The method of claim 38, further comprising contacting the particular antibody to an NK cell and measuring activation of the contacted NK cell.

40. The method of claim 38 or 39, wherein the protein is selected from the group consisting of Natural Cytotoxicity Triggering Receptor 1 (NCR1), NCR3, and CD-16.
